SEQUENCE LISTING (17 sequences)

SEQ ID NO: 1 - DNA, 8 bp, Artificial Sequence
  Description of Artificial Sequence Synthetic oligonucleotide
tggcagct

SEQ ID NO: 2 - DNA, 9 bp, Artificial Sequence
  Description of Artificial Sequence Synthetic oligonucleotide
ctggcagct

SEQ ID NO: 3 - DNA, 10 bp, Artificial Sequence
  Description of Artificial Sequence Synthetic oligonucleotide
actggcagct

SEQ ID NO: 4 - DNA, 11 bp, Artificial Sequence
  Description of Artificial Sequence Synthetic oligonucleotide
aactggcagct

SEQ ID NO: 5 - DNA, 12 bp, Artificial Sequence
  Description of Artificial Sequence Synthetic oligonucleotide
caactggcagct

SEQ ID NO: 6 - protein, 7 aa, Artificial Sequence
  Description of Artificial Sequence Synthetic peptide
GVITRIR

SEQ ID NO: 7 - protein, 9 aa, Artificial Sequence
  Description of Artificial Sequence Synthetic peptide
CKGGRAKDC

SEQ ID NO: 8 - protein, 9 aa, Artificial Sequence
  Description of Artificial Sequence Synthetic peptide
RRRRRRRRR

SEQ ID NO: 9 - protein, 4 aa, Artificial Sequence
  Description of Artificial Sequence Synthetic peptide
KKKK

SEQ ID NO: 10 - RNA, 22 nt, Homo sapiens
aagcugccaguugaagaacugu

SEQ ID NO: 11 - DNA, 22 bp, Artificial Sequence
  Description of Artificial Sequence Synthetic oligonucleotide
acagttcttcaactggcagctt

SEQ ID NO: 12 - DNA, 18 bp, Artificial Sequence
  Description of Artificial Sequence Synthetic oligonucleotide
cttcttcaactggcagct

SEQ ID NO: 13 - DNA, 13 bp, Artificial sequence
  Description of Artificial Sequence Synthetic peptide
tcaactggcagct

SEQ ID NO: 14 - DNA, 14 bp, Artificial sequence
  Description of Artificial Sequence Synthetic peptide
ttcaactggcagct

SEQ ID NO: 15 - DNA, 15 bp, Artificial sequence
  Description of Artificial Sequence Synthetic peptide
cttcaactggcagct

SEQ ID NO: 16 - DNA, 16 bp, Artificial sequence
  Description of Artificial Sequence Synthetic peptide
tcttcaactggcagct

SEQ ID NO: 17 - DNA, 17 bp, Artificial sequence
  Description of Artificial Sequence Synthetic peptide
ttcttcaactggcagct

(19) United States
(12) Patent Application Publication
(10) Pub. No.: US 2022/0056443 A1
(43) Pub. Date: Feb. 24, 2022

(54) METABOLIC BENEFITS OF SHORT MIR-22 MIRNA ANTAGOMIR THERAPIES

(71) Applicant: AptamiR Therapeutics, Inc., Naples, FL (US)

(72) Inventor: Marc THIBONNIER, Naples, FL (US)

(73) Assignee: AptamiR Therapeutics, Inc., Naples, FL (US)

(21) Appl. No.: 17/250,945

(22) PCT Filed: Sep. 6, 2019
(86) PCT No.: PCT/US2019/049855

§ 371 Date: Mar. 31, 2021

Related U.S. Application Data
(60) Provisional application No. 62/810,185, filed on Feb. 25, 2019.
(60) Provisional application No. 62/727,870, filed on Sep. 6, 2018.

(51) Int. Cl. C12N 15/113; C07K 14/00

(52) U.S. Cl. CPC C12N 15/113; C07K 14/003; C12N 2310/3341; C12N 2310/315; C12N 2310/312; C12N 2310/321; C12N 2310/3231; C12N 2310/113; C12N 2310/3513; C12N 2310/314; C12N 2310/3233; C12N 2320/30; C12N 2310/322

(57) ABSTRACT

The methods and compositions of the disclosure provide for novel therapeutic compounds to treat obesity and aspects related thereto. Embodiments of the disclosure relate to oligonucleotide therapeutic (ONT) agents targeting miR-22 miRNA for the treatment of human obesity and related cardiometabolic disorders. Accordingly, aspects of the disclosure relate to modified nucleic acids, including locked nucleic acids, ethylene-bridged nucleotides, peptide nucleic acids, phosphorodiamidate morpholino oligonucleotides, and or a 5′(E)-vinyl-phosphonate modification.

### CROSS-REFERENCE TO RELATED APPLICATIONS

This application claims the benefit of priority of U.S. Provisional Patent Application No. 62/727,870 filed Sep. 6, 2018, which is hereby incorporated by reference in its entirety.

### FIELD OF THE INVENTION

This invention relates to the fields of molecular biology, pharmacology, and synthetic organic chemistry of oligonucleotide therapeutic (ONT) agents for the treatment of human obesity and related cardiometabolic disorders including insulin resistance, type 2 diabetes mellitus, dyslipidemia, fatty infiltration of the liver and chronic inflammation.

### BACKGROUND

Human obesity has become a worldwide pandemic due to sedentary lifestyle and excessive consumption of energy-dense foods rich in saturated fats and sugars [1]. Obesity affects all ages and socioeconomic groups. The World Health Organization estimated that in 2008, 1.5 billion adults aged 20 years and older were overweight and over 500 million men and women were obese [2, 3]. These numbers are estimated to increase to 2.16 billion overweight and 1.12 billion obese individuals by 2030. Obesity is the source of lost earnings, restricted activity days, absenteeism, lower productivity at work (presenteeism), reduced quality of life, permanent disability, significant morbidity and mortality, and shortened lifespan [4]. A recent analysis of data from 195 countries revealed that the prevalence of obesity has doubled in more than 70 countries since 1980, with high BMI accounting for 4 million deaths globally [5]. An estimated 35.8 million (2.3%) of global Disability-Adjusted Life Years (DALYs) are caused by overweight or obesity.

The total annual economic cost of overweight and obesity in the United States and Canada caused by medical costs, excess mortality and disability was estimated to be about $300 billion in 2009. International studies on the economic costs of obesity have shown that they account for between 2% and 10% of total health care costs [6]. Furthermore, voluntary spending on weight loss, mostly on unproven means, exceeds $75 billion annually.

Obesity is the result of a chronic imbalance between energy intake and expenditure. This imbalance leads to storage of excess energy in the form of triglycerides in adipocytes, which typically exhibit both hypertrophy (increase in cell size) and hyperplasia (increase in cell number or adipogenesis). These events trigger a pro-inflammatory response in the adipose tissues and surrounding macrophages, resulting in a chronic systemic inflammatory state [7]. The recent worsening of obesity is due to the combination of excessive consumption of energy-dense foods high in saturated fats and sugars as well as reduced physical activity.

The current symptomatic medical treatments of human obesity have poor benefit-to-risk profiles, do not meet patients' expectations, fail to achieve long-term therapeutic goals, mainly due to limited drug efficacy and patients' poor adherence with lifestyle changes and therapies, and are often not covered by health insurance plans [8]. Several obesity drugs have been removed from the market for safety reasons and small molecules currently in development are struggling to gain regulatory approval because of their modest short-term efficacy and unknown safety profile [9]. Presently, only restrictive and malabsorptive bariatric surgery can achieve significant long-term reduction of weight excess with some favorable cardiovascular benefits. However, bariatric surgery creates a state of chronic digestive malabsorption.

Therefore, there is a pressing need to develop an effective and safe medical treatment of human obesity and related disorders that should significantly reduce health care expenses, improve and save human lives.

Non Alcoholic Fatty Liver Disease (NAFLD) is a growing pandemic, affecting up to 31% of the adult human population worldwide, in relation with an increased prevalence of obesity and type 2 diabetes mellitus [10]. Prevalence of liver steatosis is 76% in obese patients. One hundred million Americans have a fatty liver, and most don't know it. Many will develop NAFLD, Non Alcoholic Steatohepatitis (NASH), liver fibrosis, cirrhosis and possibly end stage liver failure and liver cancer [11]. NAFLD/NASH is the major cause of chronic liver disease and it is associated with substantial morbidity and mortality in developed countries [12]. NAFLD is a fairly complex disease and a “multiple-hit” hypothesis is now proposed to explain the development of NAFLD [13]: disease and a “multiple-hit” hypothesis is now proposed to explain the development of NAFLD [13]:

First hit: Lipid accumulation (steatosis)

Second hit: inflammation, mitochondrial dysfunction and oxidative stress (steatohepatitis)

Third hit: defective hepatocyte regeneration (fibrosis)

Without any currently approved therapy, NAFLD and NASH need safe and effective treatments.

MicroRNAs (miRNAs) are small non coding RNAs that bind to complementary messenger RNAs (mRNAs) and subsequently regulate protein expression [14]. Each miRNA is evolutionarily selected to modulate the expression of gene pathways. miRNAs are synthesized as long single-stranded RNAs (pri-miRNA) that fold into hairpin loop structures (pre-miRNA). These hairpins are processed by the enzymes drosha and dicer into double-stranded mature miRNAs. The guide strand complementary to target mRNA transcripts is loaded into argonaute (AGO) proteins while the passenger strand is removed [15]. The guide strand/AGO complex then binds by sequence complementarity to targets that are typically located within 3′-untranslated regions (3′-UTR) of mRNAs.

miRNAs are attractive drug candidates for regulating cell fate decisions and improving complex diseases because the simultaneous modulation of many target genes by a single miRNA may provide effective therapies of multifactorial diseases like obesity. As a matter of fact, miRNAs are extensive regulators of adipocyte differentiation, development and function and are viable therapeutic agents for obesity. Several miRNA agonists (agomirs) and antagonists (antagomirs) are currently developed to treat various human diseases, including cardiometabolic disorders [16]. The technology platform to transform oligonucleotides into drugs has recently matured and several drugs are now in clinical development [16-18].

miRNA inhibitors (antagomirs) are single-stranded oligonucleotides that bind to complementary miRNAs through Watson-Crick base-pairing, blocking their interaction with target mRNAs. To improve the structure-activity relationship and the PK/PD profile of miRNA inhibitors, the following chemical modifications are implemented. The phosphate backbone is replaced by phosphorothioate to inhibit nuclease degradation and promote plasma protein binding, thus extending circulation time and tissue distribution. Modifications of the 2′ carbon of the sugar group (2′-Fluor, 2′-O-methyl, 2′-methoxyethyl) and Locked Nucleic Acid (LNA) conformation are also used to inhibit nuclease degradation, increase affinity to target RNAs, and blunt the immune response to foreign DNA and RNA [17]. Phosphorothioate backbone modified oligonucleotides can be administered subcutaneously in saline without additional formulation (the so called “naked miRNA inhibitors”), and generally have similar and predictable pharmacokinetics. The initial distribution is rapid, with a circulating t_{1/2} of a few hours, and a bioavailability over 90%. Delivery to the liver, kidney, adipose, spleen and bone marrow is robust, with much less found in heart and muscle.

Phosphorothioate (PS) modifications of miRNAs improve their nuclease resistance and pharmacokinetic profile. However, too many PS modifications (>9) lead to toxic effects attributed to non-specific binding to cellular membrane proteins such as glycoprotein VI [19]. Alternative oligonucleotide backbone modifications instead of PS, 2′ position modifications and linkage between the 2′ and 4′ positions have been reported to enhanced nuclease resistance, thermal stability and potency/efficacy of modified miRNAs.

Clinical applications of oligonucleotide therapeutics (ONT), including miRNA analogs, have been hindered by [20-25]:
1. in vivo instability (degradation by RNases and exonucleases)
2. capture by the reticulo-endothelial system (RES) and renal clearance
3. off-target effects (partial sequence homology with non-target mRNAs or interactions with non-nucleotide entities)
4. activation of blood platelets and the complement system
5. immunogenicity (Toll-like receptor (TLR)-mediated (TLR3, TLR7, TLR8) and non-TLR-mediated (protein kinase R (PKR) and retinoic acid inducible protein (RIG-1)).
6. Tissue accumulation (liver and kidneys).
   Finally, the intracellular penetration of miRNAs is hampered by their large size, hydrophilicity and negative charges.

Several groups of research scientists have shown that 8 to 14 nucleotides long miRNA antagomirs (the 8 to 14-mer oligonucleotides also called “micro” or “tiny” antagomirs) can exert potent activities by binding to the seed region of their targeted miRNA sequence, as reported for miR-21, miR-33, miR-92, miR-122, miR-155 and let-7 [26-28]. These shorter sequences reduce the risk of binding to mismatched sequences, thus reducing off target effects. Furthermore, fewer chemical modifications within their shorter sequences should prevent toxicity events as reported for previous generations of ONTs.

### SUMMARY

The methods and compositions of the disclosure provide for novel therapeutic compounds to treat obesity and aspects related thereto. Embodiments of the disclosure relate to oligonucleotide therapeutic (ONT) agents targeting miR-22 miRNA for the treatment of human obesity and related cardiometabolic disorders. Aspects of the disclosure relate to a modified nucleic acid comprising the sequence of TGGCAGCT (SEQ ID NO:1). In some embodiments, at least one nucleotide of the nucleic acid of SEQ ID NO:1 comprises a locked nucleotide (LNA). In some embodiments, at least one nucleotide of SEQ ID NO:1 comprises an ethylene-bridged nucleotide (ENA) modification. In some embodiments, at least one nucleotide of SEQ ID NO:1 comprises a peptide nucleic acid (PNA) modification. In some embodiments, at least one nucleotide of SEQ ID NO:1 comprises a Phosphorodiamidate Morpholino Oligonucleotide (PMO) modification. In some embodiments, SEQ ID NO:1 comprises a 5′(E)-vinyl-phosphonate (VP) modification.

In some embodiments, the nucleic acid comprises at least, at most, or exactly 1, 2, 3, 4, 5, 6, 7, or 8 (or any derivable range therein) locked nucleotides. In some embodiments, the first nucleotide (T) of SEQ ID NO:1 is locked. In some embodiments, the second nucleotide (G) of SEQ ID NO:1 is locked. In some embodiments, the third nucleotide (G) of SEQ ID NO:1 is locked. In some embodiments, the fourth nucleotide (C) of SEQ ID NO:1 is locked. In some embodiments, the fifth nucleotide (A) of SEQ ID NO:1 is locked. In some embodiments, the sixth nucleotide (G) of SEQ ID NO:1 is locked. In some embodiments, the seventh nucleotide (C) of SEQ ID NO:1 is locked. In some embodiments, the eighth nucleotide (T) of SEQ ID NO:1 is locked.

In some embodiments, the nucleic acid comprises at least, at most, or exactly 1, 2, 3, 4, 5, 6, 7, or 8 (or any derivable range therein) ethylene-bridged nucleotides. In some embodiments, the first nucleotide (T) of SEQ ID NO:1 is ethylene-bridged. In some embodiments, the second nucleotide (G) of SEQ ID NO:1 is ethylene-bridged. In some embodiments, the third nucleotide (G) of SEQ ID NO:1 is ethylene-bridged. In some embodiments, the fourth nucleotide (C) of SEQ ID NO:1 is ethylene-bridged. In some embodiments, the fifth nucleotide (A) of SEQ ID NO:1 is locked. In some embodiments, the sixth nucleotide (G) of SEQ ID NO:1 is ethylene-bridged. In some embodiments, the seventh nucleotide (C) of SEQ ID NO:1 is ethylene-bridged. In some embodiments, the eighth nucleotide (T) of SEQ ID NO:1 is ethylene-bridged.

One skilled in the art would understand that a nucleotide having a locked modification, a nucleotide having an ethylene bridge, and a nucleotide having a modified 2′ ribose is mutually exclusive. Accordingly, a nucleotide having an ethylene bridge is excluded from having a locked modification or a modified 2′ ribose. A nucleotide having a locked modification is excluded from having an ethylene bridge modification or a modified 2′ ribose. A nucleotide having a modified 2′ ribose is excluded from having a locked modification or an ethylene bridge. Therefore, defining a nucleotide as being locked is also defining the nucleotide as not having an ethylene bridge or as not having a modified 2′ ribose, such as 2′-deoxy ribose, 2′O-methyl, 2′-fluoro, or 2′-methoxyethyl modified ribose.

In some embodiments, one or more nucleotides comprises a 2′-modified ribose. In some embodiments, the nucleic acid comprises one or more 2′-deoxy-ribose nucleotides. In some embodiments, the nucleic acid comprises at least, at most, or exactly 1, 2, 3, 4, 5, 6, 7, or 8 (or any derivable range therein) 2′-deoxy-ribose nucleotides. In some embodiments, the first nucleotide (T) of SEQ ID NO:1 comprises a 2′-deoxy-ribose nucleotide. In some embodiments, the second nucleotide (G) of SEQ ID NO:1 comprises a 2′-deoxy-ribose nucleotide. In some embodiments, the third nucleotide (G) of SEQ ID NO:1 comprises a 2′-deoxy-ribose nucleotide. In some embodiments, the fourth nucleotide (C) of SEQ ID NO:1 comprises a 2′-deoxy-ribose nucleotide. In some embodiments, the fifth nucleotide (A) of SEQ ID NO:1 comprises a 2′-deoxy-ribose nucleotide. In some embodiments, the sixth nucleotide (G) of SEQ ID NO:1 comprises a 2′-deoxy-ribose nucleotide. In some embodiments, the seventh nucleotide (C) of SEQ ID NO:1 comprises a 2′-deoxy-ribose nucleotide. In some embodiments, the eighth nucleotide (T) of SEQ ID NO:1 comprises a 2′-deoxy-ribose nucleotide. In some embodiments, the nucleic acid comprises one or more 2′O-methyl-ribose nucleotides. In some embodiments, the nucleic acid comprises at least, at most, or exactly 1, 2, 3, 4, 5, 6, 7, or 8 (or any derivable range therein) 2′O-methyl-ribose nucleotides. In some embodiments, the first nucleotide (T) of SEQ ID NO:1 comprises a 2′O-methyl-ribose nucleotide. In some embodiments, the second nucleotide (G) of SEQ ID NO:1 comprises a 2′O-methyl-ribose nucleotide. In some embodiments, the third nucleotide (G) of SEQ ID NO:1 comprises a 2′O-methyl-ribose nucleotide. In some embodiments, the fourth nucleotide (C) of SEQ ID NO:1 comprises a 2′O-methyl-ribose nucleotide. In some embodiments, the fifth nucleotide (A) of SEQ ID NO:1 comprises a 2′O-methyl-ribose nucleotide. In some embodiments, the sixth nucleotide (G) of SEQ ID NO:1 comprises a 2′O-methyl-ribose nucleotide. In some embodiments, the seventh nucleotide (C) of SEQ ID NO:1 comprises a 2′O-methyl-ribose nucleotide. In some embodiments, the eighth nucleotide (T) of SEQ ID NO:1 comprises a 2′O-methyl-ribose nucleotide. In some embodiments, the nucleic acid comprises one or more 2′-fluoro-ribose nucleotides. In some embodiments, the nucleic acid comprises at least, at most, or exactly 1, 2, 3, 4, 5, 6, 7, or 8 (or any derivable range therein) 2′-fluoro-ribose nucleotides. In some embodiments, the first nucleotide (T) of SEQ ID NO:1 comprises a 2′-fluoro-ribose nucleotide. In some embodiments, the second nucleotide (G) of SEQ ID NO:1 comprises a 2′-fluoro-ribose nucleotide. In some embodiments, the third nucleotide (G) of SEQ ID NO:1 comprises a 2′-fluoro-ribose nucleotide. In some embodiments, the fourth nucleotide (C) of SEQ ID NO:1 comprises a 2′-fluoro-ribose nucleotide. In some embodiments, the fifth nucleotide (A) of SEQ ID NO:1 comprises a 2′-fluoro-ribose nucleotide. In some embodiments, the sixth nucleotide (G) of SEQ ID NO:1 comprises a 2′-fluoro-ribose nucleotide. In some embodiments, the seventh nucleotide (C) of SEQ ID NO:1 comprises a comprises a 2′-fluoro-ribose nucleotide. In some embodiments, the eighth nucleotide (T) of SEQ ID NO:1 comprises a comprises a 2′-fluoro-ribose nucleotide. In some embodiments, the nucleic acid comprises one or more 2′-methoxyethyl-ribose nucleotides. In some embodiments, the nucleic acid comprises at least, at most, or exactly 1, 2, 3, 4, 5, 6, 7, or 8 (or any derivable range therein) 2′-methoxyethyl-ribose nucleotides. In some embodiments, the first nucleotide (T) of SEQ ID NO:1 comprises a 2′-methoxyethyl-ribose nucleotide. In some embodiments, the second nucleotide (G) of SEQ ID NO:1 comprises a 2′-methoxyethyl-ribose nucleotide. In some embodiments, the third nucleotide (G) of SEQ ID NO:1 comprises a 2′-methoxyethyl-ribose nucleotide. In some embodiments, the fourth nucleotide (C) of SEQ ID NO:1 comprises a 2′-methoxyethyl-ribose nucleotide. In some embodiments, the fifth nucleotide (A) of SEQ ID NO: comprises a 2′-methoxyethyl-ribose nucleotide. In some embodiments, the sixth nucleotide (G) of SEQ ID NO:1 comprises a 2′-methoxyethyl-ribose nucleotide. In some embodiments, the seventh nucleotide (C) of SEQ ID NO:1 comprises a 2′-methoxyethyl-ribose nucleotide. In some embodiments, the eighth nucleotide (T) of SEQ ID NO:1 comprises a 2′-methoxyethyl-ribose nucleotide.

In some embodiments, the nucleic acid comprises one or more phosphorothioate modified nucleotides. In some embodiments, the nucleic acid comprises at least, at most, or exactly 1, 2, 3, 4, 5, 6, or 7 (or any derivable range therein) phosphorothioate modified nucleotides. In some embodiments, the first nucleotide (T) of SEQ ID NO:1 comprises a phosphorothioate modified nucleotide. In some embodiments, the second nucleotide (G) of SEQ ID NO:1 comprises a phosphorothioate modified nucleotide. In some embodiments, the third nucleotide (G) of SEQ ID NO:1 comprises a phosphorothioate modified nucleotide. In some embodiments, the fourth nucleotide (C) of SEQ ID NO:1 comprises a phosphorothioate modified nucleotide. In some embodiments, the fifth nucleotide (A) of SEQ ID NO:1 comprises a phosphorothioate modified nucleotide. In some embodiments, the sixth nucleotide (G) of SEQ ID NO:1 comprises a phosphorothioate modified nucleotide. In some embodiments, the seventh nucleotide (C) of SEQ ID NO:1 comprises a phosphorothioate modified nucleotide.

In some embodiments, the nucleic acid comprises one or more modified nucleobases. In some embodiments, the modified nucleobase comprises one or two modified cytosines. In some embodiments, the nucleic acid comprises two modified cytosines. In some embodiments, the modified cytosine(s) comprise 5′-methylcytosine (5MeC).

In some embodiments, the nucleic acid comprises a modified terminal phosphate. In some embodiments, the modified terminal phosphate comprises 5′(E)-vinyl-phosphonate (VP).

In some embodiments, the nucleic acid comprises lnTlnGGCAGlnClnT, wherein lnX indicates that the nucleotide (X) is locked. In some embodiments, the nucleic comprises lnTlnGGCAGlnClnT and one or more further modifications as described herein. In some embodiments, the nucleic acid comprises lnTlnGdGdCdAdGlnClnT, wherein lnX indicates that the nucleotide (X) is locked and dX indicates a nucleotide (X) with a 2′-deoxy-ribose. In some embodiments, the nucleic acid comprises lnTlnGdGdCdAdGlnClnT and one or more further modifications as described herein. In some embodiments, the nucleic acid comprises lnTlnGlnGlnClnAlnGlnClnT, wherein lnX indicates that the nucleotide (X) is locked. In some embodiments, the nucleic acid compriseslnTlnGlnGlnClnAlnGlnClnT and one or more further modifications as described herein. In some embodiments, the nucleic acid comprises T*G*GCAG*C*T, wherein X* indicates a phosphorothioate modified nucleotide (X). In some embodiments, the nucleic acid comprises T*G*GCAG*C*T and one or more further modifications as described herein. In some embodiments, the nucleic acid comprises lnT*lnG*lnGlnClnAlnG*lnC*lnT, wherein X* indicates a phosphorothioate modified nucleotide (X) and lnX indicates that the nucleotide (X) is locked. In some embodiments, the nucleic acid comprises lnT*lnG*lnGlnClnAlnG*lnC*lnT and one or more further modifications as described herein. In some embodiments, the nucleic acid comprises T*G*G*C*A*G*C*T, wherein X* indicates a phosphorothioate modified nucleotide (X). In some embodiments, the nucleic acid comprises T*G*G*C*A*G*C*T and one or more further modifications as described herein. In some embodiments, the nucleic acid comprises lnT*lnG*lnG*lnC*lnAlnG*lnC*lnT, wherein X* indicates a phosphorothioate modified nucleotide (X) and lnX indicates that the nucleotide (X) is locked. In some embodiments, the nucleic acid comprises lnT*lnG*lnG*lnC*lnAlnG*lnC*lnT and one or more further modifications as described herein. In some embodiments, the nucleic acid comprises lnT*lnG*lnGlnMeClnAlnG*ln5MeC*lnT, wherein X* indicates a phosphorothioate modified nucleotide (X), lnX indicates that the nucleotide (X) is locked, and 5MeC indicates a 5-methylcytosine nucleotide. In some embodiments, the nucleic acid comprises lnT*lnG*lnGln5MeClnAlnG*ln5MeC*lnT and one or more further modifications as described herein. In some embodiments, the nucleic acid comprises lnT*lnG*dGdCdAdG*lnC*lnT, wherein lnX indicates that the nucleotide (X) is locked, X* indicates a phosphorothioate modified nucleotide (X), and dX indicates a nucleotide (X) with a 2′-deoxy-ribose. In some embodiments, the nucleic acid comprises lnT*lnG*dGdCdAdG*lnC*lnT and one or more further modifications as described herein. In some embodiments, the nucleic acid comprises lnT*lnG*dG*dC*dA*dG*lnC*lnT, wherein lnX indicates that the nucleotide (X) is locked, X* indicates a phosphorothioate modified nucleotide (X), and dX indicates a nucleotide (X) with a 2′-deoxy-ribose. In some embodiments, the nucleic acid comprises lnT*lnG*dG*dC*dA*dG*lnC*lnT and one or more further modifications as described herein. In some embodiments, the nucleic acid comprises dT*lnG*dG*ln5MeC*lnA*dG*ln5MeC*lnT, wherein lnX indicates that the nucleotide (X) is locked, 5MeC indicates a 5-methylcytosine nucleotide, X* indicates a phosphorothioate modified nucleotide (X), and dX indicates a nucleotide (X) with a 2′-deoxy-ribose. In some embodiments, the nucleic acid comprises dT*lnG*dG*ln5MeClnA*dG*ln5MeClnT and one or more further modifications as described herein. In some embodiments, the nucleic acid comprises dT*lnG*dGln5MeClnAdG*ln5MeC*lnT, wherein lnX indicates that the nucleotide (X) is locked, 5MeC indicates a 5-methylcytosine nucleotide, X* indicates a phosphorothioate modified nucleotide (X), and dX indicates a nucleotide (X) with a 2′-deoxy-ribose. In some embodiments, the nucleic acid comprises dT*lnG*dGln5MeClnAdG*ln5MeC*lnT and one or more further modifications as described herein. In some embodiments, the nucleic acid comprises enTenGGCAGenCenT, wherein enX indicates that the nucleotide (X) is an ethylene-bridged nucleotide. In some embodiments, the nucleic acid comprises enTenGGCAGenCenT and one or more further modifications as described herein. In some embodiments, the nucleic acid comprises enTenGdGdCdAdGenCenT, wherein enX indicates that the nucleotide (X) is an ethylene-bridged nucleotide and dX indicates a nucleotide (X) with a 2′-deoxy-ribose. In some embodiments, the nucleic acid comprises enTenGdGdCdAdGenCenT and one or more further modifications as described herein. In some embodiments, the nucleic acid comprises the sequence of T*G*GCAG*C*T, wherein

X* indicates a phosphorothioate modified nucleotide (X). In some embodiments, the nucleic acid comprises the sequence of T*G*GCAG*C*T and one or more further modifications as described herein. In some embodiments, the nucleic acid comprises enT*enG*dGdCdAdG*enC*enT, wherein X* indicates a phosphorothioate modified nucleotide (X), enX indicates that the nucleotide (X) is an ethylene-bridged nucleotide and dX indicates a nucleotide (X) with a 2′-deoxy-ribose. In some embodiments, the nucleic acid comprises enT*enG*dGdCdAdG*enC*enT and one or more further modifications as described herein. In some embodiments, the nucleic acid comprises T*G*G*C*A*G*C*T, wherein X* indicates a phosphorothioate modified nucleotide (X). In some embodiments, the nucleic acid comprises T*G*G*C*A*G*C*T and one or more further modifications as described herein. In some embodiments, the nucleic acid comprises enT*enG*dG*dC*dA*dG*enC*enT, wherein X* indicates a phosphorothioate modified nucleotide (X), enX indicates that the nucleotide (X) is an ethylene-bridged nucleotide and dX indicates a nucleotide (X) with a 2′-deoxy-ribose. In some embodiments, the nucleic acid comprises enT*enG*dG*dC*dA*dG*enC*enT and one or more further modifications as described herein. In some embodiments, the nucleic acid comprises enT*enG*enGen5MeC)enAenG*en5MeC*enT, wherein X* indicates a phosphorothioate modified nucleotide (X) and 5MeC indicates a 5-methylcytosine nucleotide. In some embodiments, the nucleic acid comprises enT*enG*enGen5MeCenAenG*en5MeC*enT and one or more further modifications as described herein. In some embodiments, the nucleic acid comprises enTenGenGenCenAenGenCenT, wherein enX indicates that the nucleotide (X) is an ethylene-bridged nucleotide. In some embodiments, the nucleic acid comprises enTenGenGenCenAenGenCenT and one or more further modifications as described herein. In some embodiments, the nucleic acid comprises enT*enG*enGenCenAenG*enC*enT, wherein enX indicates that the nucleotide (X) is an ethylene-bridged nucleotide and X* indicates a phosphorothioate modified nucleotide (X). In some embodiments, the nucleic acid comprises enT*enG*enGenCenAenG*enC*enT and one or more further modifications as described herein. In some embodiments, the nucleic acid comprises enT*enG*enG*enC*enA*enG*enC*enT, wherein enX indicates that the nucleotide (X) is an ethylene-bridged nucleotide and X* indicates a phosphorothioate modified nucleotide (X). In some embodiments, the nucleic acid comprises enT*enG*enG*enC*enA*enG*enC*enT and one or more further modifications as described herein. In some embodiments, the nucleic acid comprises VP-lnT*lnG*dGdCdAdG*lnC*lnT, wherein lnX indicates that the nucleotide (X) is locked, X* indicates a phosphorothioate modified nucleotide (X), dX indicates a nucleotide (X) with a 2′-deoxy-ribose and VP indicates a 5′(E)-vinyl-phosphonate. In some embodiments, the nucleic acid comprises VP-lnT*lnG*dGdCdAdG*lnC*lnT and one or more further modifications as described herein. In some embodiments, the nucleic acid comprises VP-enT*enG*dGdCdAdG*enC*enT, wherein enX indicates that the nucleotide (X) is an ethylene-bridged nucleotide, X* indicates a phosphorothioate modified nucleotide (X), dX indicates a nucleotide (X) with a 2′-deoxy-ribose and VP indicates a 5′(E)-vinyl-phosphonate. In some embodiments, the nucleic acid comprises VP-enT*enG*dGdCdAdG*enC*enT and one or more further modifications as described herein. In some embodiments, the nucleic acid comprises VP-lnT*lnG*lnGln5MeClnAlnG*ln5MeC*lnT, wherein lnX indicates that the nucleotide (X) is locked, X* indicates a phosphorothioate modified nucleotide (X), 5MeC indicates a 5-methylcytosine nucleotide, and VP indicates a 5′(E)-vinyl-phosphonate. In some embodiments, the nucleic acid comprises VP-lnT*lnG*lnGln5MeClnAlnG*ln5MeC*lnT and one or more further modifications as described herein. In some embodiments, the nucleic acid comprises VP-enT*enG*enGen5MeCenAenG*en5MeC*enT, wherein enX indicates that the nucleotide (X) is an ethylene-bridged nucleotide, X* indicates a phosphorothioate modified nucleotide (X), 5MeC indicates a 5-methylcytosine nucleotide, and VP indicates a 5′(E)-vinyl-phosphonate. In some embodiments, the nucleic acid comprises VP-enT*enG*enGen5MeCenAenG*en5MeC*enT and one or more further modifications as described herein. In some embodiments, the nucleic acid comprises VP-(mT)(fG)(mG)(fC)(mA)(fG)(mC)(fT), wherein VP indicates an 5′ (E)-vinyl-phosphonate, mX indicates a nucleotide (X) with a 2′ O-methyl modified ribose, and fX indicates a nucleotide (X) with a 2′ F modified ribose. In some embodiments, the nucleic acid comprises VP-(mT)(fG)(mG)(fC)(mA)(fG)(mC)(fT) and one or more further modifications as described herein. In some embodiments, the nucleic acid comprises VP-(mT)*(fG)*(mG)(fC)(mA)(fG)*(mC)*(fT), wherein VP indicates an 5′ (E)-vinyl-phosphonate, mX indicates a nucleotide (X) with a 2′ O-methyl modified ribose, fX indicates a nucleotide (X) with a 2′ F modified ribose, and X* indicates a phosphorothioate modified nucleotide (X). In some embodiments, the nucleic acid comprises VP-(mT)*(fG)*(mG)(fC)(mA)(fG)*(mC)*(fT) and one or more further modifications as described herein. In some embodiments, the nucleic acid comprises VP-(fT)(mG)(fG)(mC)(fA)(mG)(fC)(mT), wherein VP indicates an 5′ (E)-vinyl-phosphonate, mX indicates a nucleotide (X) with a 2′ O-methyl modified ribose, and fX indicates a nucleotide (X) with a 2′ F modified ribose. In some embodiments, the nucleic acid comprises VP-(fT)(mG)(fG)(mC)(fA)(mG)(fC)(mT) and one or more further modifications as described herein. In some embodiments, the nucleic acid comprises VP-(fT)*(mG)*(fG)(mC)(fA)(mG)*(fC)*(mT), wherein VP indicates an 5′(E)-vinyl-phosphonate, mX indicates a nucleotide (X) with a 2′ O-methyl modified ribose, fX indicates a nucleotide (X) with a 2′ F modified ribose, and X* indicates a phosphorothioate modified nucleotide (X). In some embodiments, the nucleic acid comprises VP-(fT)*(mG)*(fG)(mC)(fA)(mG)*(fC)*(mT) and one or more further modifications as described herein.

Some embodiments of the disclosure relate to a peptide nucleic acid (PNA) comprising the sequence:

(SEQ ID NO: 1)
TGGCAGCT,
(SEQ ID NO: 2)
CTGGCAGCT,
(SEQ ID NO: 3)
ACTGGCAGCT,
(SEQ ID NO: 4)
AACTGGCAGCT,
or
(SEQ ID NO: 5)
CAACTGGCAGCT.

Some embodiments of the disclosure relate to a phosphorodiamidate morpholino oligonucleotide (PMO) comprising the sequence:

(SEQ ID NO: 1)
TGGCAGCT,
(SEQ ID NO: 2)
CTGGCAGCT,
(SEQ ID NO: 3)
ACTGGCAGCT,
(SEQ ID NO: 4)
AACTGGCAGCT,
or
(SEQ ID NO: 5)
CAACTGGCAGCT.

In some embodiments, the peptide nucleic acid or the phosphorodiamidate morpholino oligonucleotide further comprise a modification at the carboxy and/or amino terminus of the peptide nucleic acid or the phosphorodiamidate morpholino oligonucleotide. In some embodiments, the modification comprises one or more conjugated molecules. In some embodiments, the conjugated molecule(s) comprise one or more of Hexarelin, a TSP-1 peptide comprising the amino acid sequence GVITRIR (SEQ ID NO:6), a PHB peptide comprising the amino acid sequence CKGGRAKDC (SEQ ID NO:7), a 9R peptide comprising the amino acid sequence RRRRRRRRR (SEQ ID NO:8), a 4K peptide comprising the amino acid sequence KKKK (SEQ ID NO:9), or to polyethylenimine. In some embodiments, the modification is conjugated to the carboxy terminus.

Hexarelin has the following chemical structure and name:

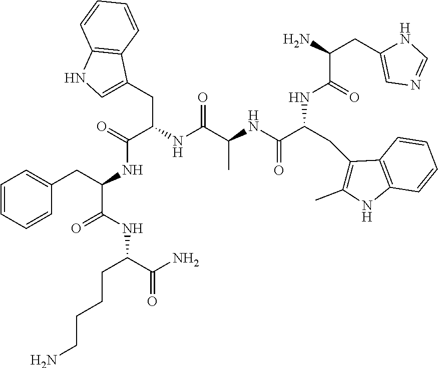 [SMILES: Cc1[nH]c2ccccc2c1C[C@@H](NC(=O)[C@@H](N)Cc1cnc[nH]1)C(=O)N[C@@H](C)C(=O)N[C@@H](Cc1c[nH]c2ccccc12)C(=O)N[C@H](Cc1ccccc1)C(=O)N[C@@H](CCCCN)C(N)=O]

L-Histidyl-2-methyl-D-tryptophyl-L-alanyl-L-tryptophyl-D-phenylalanyl-L-lysinamide.

In some embodiments, the peptide nucleic acid or the phosphorodiamidate morpholino oligonucleotide comprise Hexarelin conjugated to the carboxy terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise a TSP-1 peptide conjugated to the carboxy terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise a PHB peptide conjugated to the carboxy terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprises 9R peptide (SEQ ID NO: 8) conjugated to the carboxy terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise a 4K peptide (SEQ ID NO: 9) conjugated to the carboxy terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise polyethylenimine conjugated to the carboxy terminus of the peptide nucleic acid or the phosphorodiamidate morpholino oligonucleotide of TGGCAGCT (SEQ ID NO:1). In some embodiments, the modification is conjugated to the amino terminus. In some embodiments, the peptide nucleic acid or the phosphorodiamidate morpholino oligonucleotide comprise Hexarelin conjugated to the amino terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise a TSP-1 peptide conjugated to the amino terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise a PHB peptide conjugated to the amino terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise a 9R peptide (SEQ ID NO: 8) conjugated to the amino terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise a 4K peptide (SEQ ID NO: 9) conjugated to the amino terminus of the peptide nucleic acid or the phosphorodiamidate morpholino oligonucleotide of TGGCAGCT (SEQ ID NO:1). In some embodiments, the peptide nucleic acid or the phosphorodiamidate morpholino oligonucleotide comprise polyethylenimine conjugated to the amino terminus of the peptide nucleic acid or the phosphorodiamidate morpholino oligonucleotide of TGGCAGCT (SEQ ID NO:1).

In some embodiments, the peptide nucleic acid or the phosphorodiamidate morpholino oligonucleotide comprise Hexarelin conjugated to the carboxy terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprise a TSP-1 peptide conjugated to the carboxy terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprise a PHB peptide conjugated to the carboxy terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprise a 9R peptide (SEQ ID NO: 8) conjugated to the carboxy terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprises a 4K peptide (SEQ ID NO: 9) conjugated to the carboxy terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprises polyethylenimine conjugated to the carboxy terminus of the peptide nucleic acid or the phosphorodiamidate morpholino oligonucleotide of CTGGCAGCT (SEQ ID NO:2). In some embodiments, the modification is conjugated to the amino terminus. In some embodiments, the peptide nucleic acid or the phosphorodiamidate morpholino oligonucleotide comprise Hexarelin conjugated to the amino terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprise a TSP-1 peptide conjugated to the amino terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprise a PHB peptide conjugated to the amino terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprise a 9R peptide (SEQ ID NO: 8) conjugated to the amino terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprise a 4K peptide (SEQ ID NO: 9) conjugated to the amino terminus of the peptide nucleic acid or the phosphorodiamidate morpholino oligonucleotide of CTGGCAGCT (SEQ ID NO:2). In some embodiments, the peptide nucleic acid or the phosphorodiamidate morpholino oligonucleotide comprise polyethylenimine conjugated to the amino terminus of the peptide nucleic acid or the phosphorodiamidate morpholino oligonucleotide of CTGGCAGCT (SEQ ID NO:2).

In some embodiments, the peptide nucleic acid or the phosphorodiamidate morpholino oligonucleotide comprise Hexarelin conjugated to the carboxy terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprise a TSP-1 peptide conjugated to the carboxy terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprise a PHB peptide conjugated to the carboxy terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprise a 9R peptide (SEQ ID NO: 8) conjugated to the carboxy terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprises a 4K peptide (SEQ ID NO: 9) conjugated to the carboxy terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprises polyethylenimine conjugated to the carboxy terminus of the peptide nucleic acid or the phosphorodiamidate morpholino oligonucleotide of ACTGGCAGCT (SEQ ID NO:3). In some embodiments, the modification is conjugated to the amino terminus. In some embodiments, the peptide nucleic acid or the phosphorodiamidate morpholino oligonucleotide comprises Hexarelin conjugated to the amino terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprise a TSP-1 peptide conjugated to the amino terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprise a PHB peptide conjugated to the amino terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprise a 9R peptide (SEQ ID NO: 8) conjugated to the amino terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprises a 4K peptide (SEQ ID NO: 9) conjugated to the amino terminus of the peptide nucleic acid or the phosphorodiamidate morpholino oligonucleotide of ACTGGCAGCT (SEQ ID NO:3). In some embodiments, the peptide nucleic acid or the phosphorodiamidate morpholino oligonucleotide comprise polyethylenimine conjugated to the amino terminus of the peptide nucleic acid or the phosphorodiamidate morpholino oligonucleotide of ACTGGCAGCT (SEQ ID NO:3).

In some embodiments, the peptide nucleic acid or the phosphorodiamidate morpholino oligonucleotide comprise Hexarelin conjugated to the carboxy terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprise a TSP-1 peptide conjugated to the carboxy terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprise a PHB peptide conjugated to the carboxy terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprise a 9R peptide (SEQ ID NO: 8) conjugated to the carboxy terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprises a 4K peptide (SEQ ID NO: 9) conjugated to the carboxy terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprise polyethylenimine conjugated to the carboxy terminus of the peptide nucleic acid or the phosphorodiamidate morpholino oligonucleotide of AACTGGCAGCT (SEQ ID NO:4). In some embodiments, the modification is conjugated to the amino terminus. In some embodiments, the peptide nucleic acid or the phosphorodiamidate morpholino oligonucleotide comprise Hexarelin conjugated to the amino terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprises a TSP-1 peptide conjugated to the amino terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprise a PHB peptide conjugated to the amino terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprise a 9R peptide (SEQ ID NO: 8) conjugated to the amino terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprise a 4K peptide (SEQ ID NO: 9) conjugated to the amino terminus of the peptide nucleic acid or the phosphorodiamidate morpholino oligonucleotide of AACTGGCAGCT (SEQ ID NO:4). In some embodiments, the peptide nucleic acid or the phosphorodiamidate morpholino oligonucleotide comprise polyethylenimine conjugated to the amino terminus of the peptide nucleic acid or the phosphorodiamidate morpholino oligonucleotide of AACTGGCAGCT (SEQ ID NO:4).

In some embodiments, the peptide nucleic acid or the phosphorodiamidate morpholino oligonucleotide comprise Hexarelin conjugated to the carboxy terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise a TSP-1 peptide conjugated to the carboxy terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise a PHB peptide conjugated to the carboxy terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise a 9R peptide (SEQ ID NO: 8) conjugated to the carboxy terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise a 4K peptide (SEQ ID NO: 9) conjugated to the carboxy terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise polyethylenimine conjugated to the carboxy terminus of the peptide nucleic acid or the phosphorodiamidate morpholino oligonucleotide of CAACTGGCAGCT (SEQ ID NO:5). In some embodiments, the modification is conjugated to the amino terminus. In some embodiments, the peptide nucleic acid or the phosphorodiamidate morpholino oligonucleotide comprise Hexarelin conjugated to the amino terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise a TSP-1 peptide conjugated to the amino terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise a PHB peptide conjugated to the amino terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise a 9R peptide (SEQ ID NO: 8) conjugated to the amino terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise a 4K peptide (SEQ ID NO: 9) conjugated to the amino terminus of the peptide nucleic acid or the phosphorodiamidate morpholino oligonucleotide of CAACTGGCAGCT (SEQ ID NO:5). In some embodiments, the peptide nucleic acid or the phosphorodiamidate morpholino oligonucleotide comprise polyethylenimine conjugated to the amino terminus of the peptide nucleic acid or the phosphorodiamidate morpholino oligonucleotide of CAACTGGCAGCT (SEQ ID NO:5).

In some embodiments, the nucleic acid molecule or peptide nucleic acid or the phosphorodiamidate morpholino oligonucleotide described herein are conjugated or further conjugated to a fatty acid. In some embodiments, the nucleic acid is conjugated to a fatty acid at the 5′ end of the nucleic acid. In some embodiments, the nucleic acid is conjugated to a fatty acid at the 3′ end of the nucleic acid. In some embodiments, the peptide nucleic acid is conjugated to a fatty acid at the carboxy terminus of the molecule. In some embodiments, the peptide nucleic acid is conjugated to a fatty acid at the amino terminus of the molecule. In some embodiments, the phosphorodiamidate morpholino oligonucleotide is conjugated to a fatty acid at the carboxy terminus of the molecule. In some embodiments, the phosphorodiamidate morpholino oligonucleotide is conjugated to a fatty acid at the amino terminus of the molecule. In some embodiments, the fatty acid is a C10-35 chain fatty acid. In some embodiments, the fatty acid comprises decanoic acid, dodecanoic acid, oleic acid, stearic acid, docosanoic acid, or dotriacontahexaenoic acid. Further embodiments are described in WO2017187426, which is herein incorporated by reference for all purposes.

Method aspects of the disclosure relate to a method for antagonizing or inhibiting miR-22 in a cell or subject, the method comprising administering a nucleic acid or a peptide nucleic acid or a phosphorodiamidate morpholino oligonucleotide of the disclosure to the cell or subject. In some embodiments, miR-22-3p is specifically antagonized or inhibited. Further method aspects relate to any one or a combination of: a method for increasing lipid oxidation and thermogenesis, a method of affecting weight loss in a subject, a method of increasing caloric expenditure in a subject, a method of decreasing total fat mass in a subject, a method of decreasing blood glucose levels in a subject, a method of decreasing blood insulin levels in a subject, a method of maintaining insulin sensitivity in a subject, a method for reducing A1C in a subject, a method for converting white adipocytes to brown adipocytes in a subject, a method for increasing lipolysis in a subject, a method for increasing beta-oxidation of fatty acids in a subject, and/or a method for treating diabetes mellitus in a subject.

Further aspects of the disclosure relate to a method for treating non-alcoholic fatty liver disease (NAFLD) and/or metabolic disorders associated with NAFLD in a subject comprising administering a nucleic acid or a peptide nucleic acid or a phosphorodiamidate morpholino oligonucleotide of the disclosure to the subject. In some embodiments, the metabolic disorder is a NALFD-associated metabolic disorder. In some embodiments, the metabolic disorder comprises one or more of liver steatosis, liver inflammation, liver fibrosis, hepatocellular mitochondrial dysfunction, and hepatocellular apoptosis.

Further aspects of the disclosure relate to a method for treating one or more of liver steatosis (non-alcoholic fatty liver disease (NAFLD) and steatohepatitis (NASH)), liver inflammation, liver fibrosis, hepatocellular mitochondrial dysfunction, and hepatocellular apoptosis in a subject, the method comprising administering a nucleic acid or a peptide nucleic acid of the disclosure to the subject.

Further aspects of the disclosure relate to a method for treating or preventing cirrhosis, chronic liver disease, and/or hepatocellular carcinoma in a subject, the method comprising administering a nucleic acid or a peptide nucleic acid of the disclosure to the subject. In some embodiments, the method comprising reducing the risk of contracting one or more of cirrhosis, chronic liver disease, and/or hepatocellular carcinoma through administration of a nucleic acid or a peptide nucleic acid of the disclosure.

In some embodiments, the method comprises or further comprises treatment with one or more additional agents. In some embodiments, the one or more additional agents comprise one or more of metformin, insulin sensitizers, thyroid hormone receptor agonists, GLP-1 analogs, peroxisomal proliferator activated receptors gamma agonists, peroxisomal proliferator activated receptors alpha/delta agonists, antilipidemic drugs, polyunsaturated FA, antioxidants agents, anti-inflammatory agents, probiotics, cytoprotective agents, antiapoptotic agents, renin-angiotensin-aldosterone system agents, and vitamin D, nutraceuticals, bile acid receptors (FXR) agents, monoclonal antibodies, and ligand conjugated antisense drugs. In some embodiments, the one or more additional agents comprise one or more therapeutic agents described herein.

In embodiments of any of the disclosed methods, the subject is a human. In further embodiments of any of the disclosed methods, the subject is overweight or obese or has a genetic or epigenetic predisposition to obesity. In some embodiments of any of the disclosed methods, the nucleic acid or peptide nucleic acid or phosphorodiamidate morpholino oligonucleotide is linked to a targeting moiety. In some embodiments, the targeting moiety delivers the miRNA agent to a specific cell type, organ or tissue. In some embodiments, the diabetes comprises type 2 diabetes mellitus. In some embodiments, the type 2 diabetes mellitus is early onset type 2 diabetes mellitus. In some embodiments of the disclosed methods, a therapeutically effective amount is administered to the subject.

In some embodiments, the nucleic acid or peptide nucleic acid or phosphorodiamidate morpholino oligonucleotide is delivered trans-dermally to the subject.

A “disease” is defined as a pathological condition of a body part, an organ, or a system resulting from any cause, such as infection, genetic defect, or environmental stress. A “health-related condition” is defined herein to refer to a condition of a body part, an organ, or a system that may not be pathological, but for which treatment is sought.

As used herein, the phrases “treating and/or preventing” or “treatment and/or prevention” includes the administration of the compositions, compounds or agents of the invention to prevent or delay the onset of the symptoms, complications, or biochemical indicia of a disease, alleviating or ameliorating the symptoms or arresting or inhibiting further development of the disease, condition, or disorder (e.g., cancer). “Treating and/or preventing” further refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the phrase “treating and/or preventing” includes the administration of the therapeutic agents of the disclosure to prevent or delay, to alleviate, or to arrest or inhibit development of the symptoms or conditions associated with a disease or condition described herein.

A “therapeutically effective amount” of a substance/molecule may vary according to factors such as the disease state, age, sex, and weight of the individual, and the ability of the substance/molecule to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, the methods and systems of the present invention that “comprises,” “has,” “includes” or “contains” one or more elements possesses those one or more elements but is not limited to possessing only those one or more elements. Likewise, an element of a method or system of the present invention that “comprises,” “has,” “includes” or “contains” one or more features possesses those one or more features but is not limited to possessing only those one or more features.

As used herein the term “consisting essentially of” refers to those elements required for a given embodiment. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active.

Any method or system of the present invention can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

Throughout this application, the term “about” is used to indicate that a value includes the standard deviation of error for the device and/or method being employed to determine the value.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

As used herein, in the specification, “a” or “an” may mean one or more, unless clearly indicated otherwise. As used herein, in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic representation of the chemical modifications of oligonucleotide backbone, sugars, bases and the 5′-phosphate introduced to optimize specificity, activity, pharmacokinetic/pharmacodynamic profile and safety of ONTs [29].

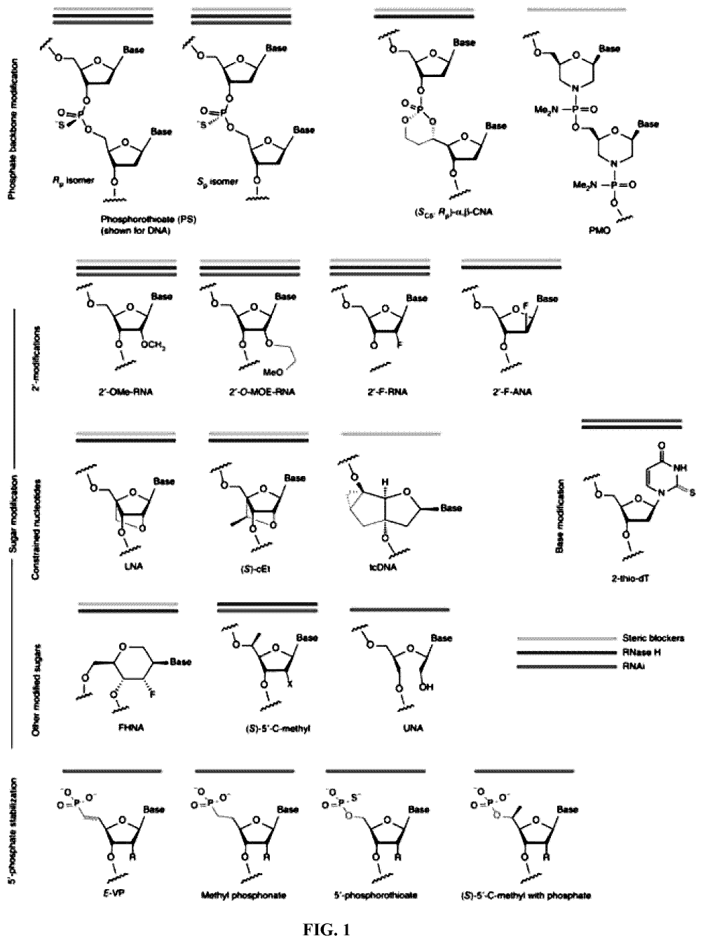

FIG. 2 is a schematic representation of the mechanism of action of short miRNA antagomirs targeting the seed region of their complementary miRNA target [26]. (A): typical Argonaute (AGO)-associated miRNA binding to complementary sequence of target mRNA. (B): example of a short miRNA antagomir designed to complementary bind to the seed region of a target miRNA. (C): high affinity binding of the short miRNA antagomir (Tiny LNA) to the target miRNA prevents the association of this miRNA with its mRNA targets, thus leading to de-repression of the target mRNAs expression.

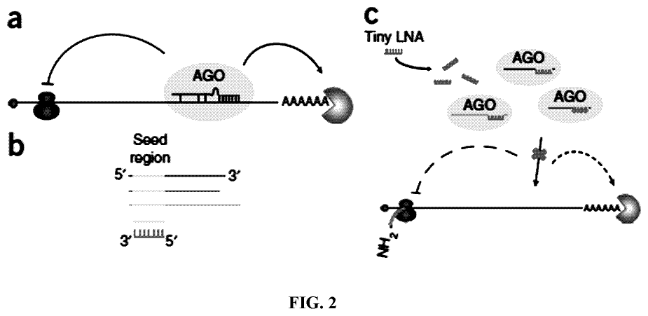

FIG. 3 is a schematic representation of the mitochondrial respiratory chain complexes producing ATP (top graph) and Uncoupling of complexes IV and V by UCP1 in adipose tissue leading to thermogenesis/energy wasting (bottom graph) [30].

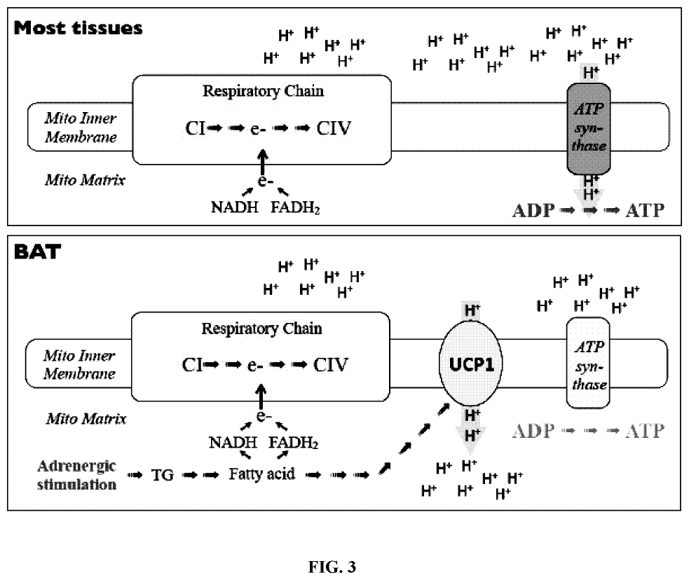

FIG. 4 depicts the In vitro protocol validation of micro antagomir candidates in primary cultures of human subcutaneous adipocytes.

FIG. 5 depicts the browning effect (size reduction of intra-cellular lipid droplets) of specific miR-22-3p 8-mer micro antagomirs in human subcutaneous adipocytes.

FIG. 6 summarizes the target genes involved in lipid oxidation, mitochondrial functions, thermogenesis, glucose metabolism, adipocyte differentiation, inflammation and anti-oxidation modulated by miR-22-3p.

FIG. 7 illustrates the reduction of fatty liver infiltration after 3 months of treatment with a miR-22-3p antagomir (H&E staining).

### DETAILED DESCRIPTION

MicroRNAs (miRNAs) are small noncoding RNAs that bind to complementary messenger RNAs (mRNAs) and subsequently regulate protein expression. Each miRNA is evolutionarily selected to modulate the expression of gene pathways. miRNAs are synthesized as long single-stranded RNAs (pri-miRNA) that fold into hairpin loop structures (pre-miRNA). These hairpins are processed by the enzymes drosha and dicer into double-stranded mature miRNAs. The guide strand complementary to target mRNA transcripts is loaded into argonaute (AGO) proteins while the passenger strand is removed [15]. The guide strand/AGO complex then binds by sequence complementarity to targets that are typically located within 3′-untranslated regions (3′-UTR) of mRNAs.

miRNAs are attractive drug candidates for regulating cell fate decisions and improving complex diseases because the simultaneous modulation of many target genes by a single miRNA may provide effective therapies of multifactorial diseases like obesity. miRNA inhibitors (antagomirs) are single-stranded oligonucleotides that bind to complementary miRNAs through Watson-Crick base-pairing, blocking their interaction with target mRNAs. The disclosed nucleic acids and peptide nucleic acids of the disclosure can be used as antagomirs, and the modifications may improve the structure-activity relationship of miRNA inhibitors.

The current disclosure describes compositions and methods using antagomirs that transform fat storing adipocytes into fat burning adipocytes. The inventor has demonstrated that miR-22 inhibition by a miR-22-3p antagomir can efficiently treat human obesity and type 2 diabetes by increasing lipid oxidation, mitochondrial activity and energy expenditure. Genes like KDM3A, KDM6B, PPARA, PPARGC1B and SIRT1 involved in lipid catabolism, thermogenesis and glucose homeostasis are conserved targets for miR-22-3p. In primary cultures of human subcutaneous adipocytes, miR-22-3p inhibition modified target genes expression and increased lipid oxidation, mitochondrial activity and energy expenditure. In a model of Diet-Induced Obesity in mice of various ages, weekly subcutaneous injections of miR22-3p inhibitors for up to twelve weeks produced an increase of energy expenditure with fat mass reduction, no appetite reduction nor body temperature increase. Insulin sensitivity as well as circulating glucose, cholesterol and leptin were also improved. Oral glucose tolerance tests were normalized. There was a dramatic reduction of liver steatosis at completion of the study after 3 months of active treatment. RNA sequencing by NGS revealed an activation of lipid metabolism pathways. These findings demonstrate that microRNA-22-3p inhibition could lead to a potent treatment of fat accumulation and related complex metabolic disorders like obesity and type 2 diabetes mellitus (the so-called diabesity) as well as Non Alcoholic Fatty Liver Disease.

Clinical applications of oligonucleotide therapeutics (ONT), including miRNA analogs, have been hindered by in vivo instability (degradation by RNases and exonucleases), capture by the reticulo-endothelial system (RES) and renal clearance, off-target effects (partial sequence homology with non-target mRNAs or interactions with non-nucleotide entities), activation of blood platelets and the complement system, immunogenicity (Toll-like receptor (TLR)-mediated (TLR3, TLR7, TLR8) and non-TLR-mediated (protein kinase R (PKR) and retinoic acid inducible protein (RIG-1)), tissue accumulation (liver and kidneys), and insufficient intracellular penetration due to size, hydrophilicity, and/or negative charges.

The inventors hypothesized that potent inhibition of a specific miRNA target could be achieved by shorter miRNA antagomirs that specifically bind to the seed region of their complementary miRNA, thus preventing the interaction of the targeted miRNA with the RISC complex. Accordingly, several antagomirs were designed to solve the aforementioned problems.

### I. OLIGONUCLEOTIDES

The term “nucleoside” refers to a unit made up of a heterocyclic base and its sugar. The term “nucleotide” refers to a nucleoside having a phosphate group on its 3′ or 5′ sugar hydroxyl group. The term “Oligonucleotide” refers to a plurality of joined nucleotide units formed in a specific sequence from naturally occurring bases and pentofuranosyl groups joined through a sugar group by native phosphodiester bonds. This term refers to both naturally occurring and synthetic species formed from naturally occurring subunits.

The presently disclosed compounds generally can be viewed as “oligonucleotide analogs,” that is, compounds which function like oligonucleotides, but which have non-naturally occurring portions. Oligonucleotide analogs can have altered sugar moieties, altered base moieties or altered inter-sugar linkages. The term “oligomers” is intended to encompass oligonucleotides, oligonucleotide analogs or oligonucleosides. Thus, in speaking of “oligomers” reference is made to a series of nucleosides or nucleoside analogs that are joined via either natural phosphodiester bonds or other linkages, including the four atom linkers. Although the linkage generally is from the 3′ carbon of one nucleoside to the 5′ carbon of a second nucleoside, the term “oligomer” can also include other linkages such as 2′-5′ linkages.

Oligonucleotide analogs also can include other modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true nucleic acid species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound. Preferred linkages have structure CH2-RA-NR1 CH2, CH2-NR1-RA-CH2, RA-NR1-CH2-CH2, CH2-CH2-NR1-RA, CH2-CH2-RA-NR1, or NR1-RA-CH2-CH2 where RA is O or NR2.

Methods for the preparation of oligonucleosides are disclosed. Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique [37]. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass [53], TentaGel Support—an aminopolyethyleneglycol derivatized support [54] or Poros—a copolymer of polystyrene/divinylbenzene. Attachment and cleavage of nucleotides and oligonucleotides can be affected via standard procedures [55]. As used herein, the term solid support further includes any linkers (e.g., long chain alkyl amines and succinyl residues) used to bind a growing oligonucleoside to a stationary phase such as CPG.

A. Locked Nucleotides

A locked nucleic acid (LNA or Ln), also referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. Such oligomers are synthesized chemically and are commercially available. The locked ribose conformation enhances base stacking and backbone pre-organization. This significantly increases the hybridization properties (melting temperature) of oligonucleotides.

B. Ethylene Bridged Nucleotides

Ethylene-bridged nucleic acids (ENA or En) are modified nucleotides with a 2′-O, 4′C ethylene linkage. Like locked nucleotides, these nucleotides also restrict the sugar puckering to the N-conformation of RNA.

C. Peptide Nucleic Acids

Peptide nucleic acids (PNA or Pn) mimic the behavior of DNA and binds complementary nucleic acid strands. The term, “peptide,” when used herein may also refer to a peptide nucleic acid. PNA is an artificially synthesized polymer similar to DNA or RNA. DNA and RNA have a deoxyribose and ribose sugar backbone, respectively, whereas PNA's backbone is composed of repeating N-(2-aminoethyl)-glycine units linked by peptide bonds. The various purine and pyrimidine bases are linked to the backbone by a methylene bridge (—CH2-) and a carbonyl group (—(C═O)—). PNAs are depicted like peptides, with the N-terminus at the first (left) position and the C-terminus at the last (right) position.

Since the backbone of PNAs contains no charged phosphate groups, the binding between PNA/DNA strands is stronger than between DNA/DNA strands due to the lack of electrostatic repulsion. PNAs are not easily recognized by either nucleases or proteases, making them resistant to degradation by enzymes. PNAs are also stable over a wide pH range. In some aspects, the PNAs described herein have improved cytosolic delivery over other PNAs.

D. Phosphorodiamidate Morpholino Oligonucleotides

Phosphorodiamidate morpholino oligomers (PMO or Po) are short single-stranded DNA analogs that are built upon a backbone of morpholine rings connected by phosphorodiamidate linkages. PMOs are uncharged nucleic acid analogs that are less likely to interact with proteins. PMOs bind to complementary sequences of target mRNAs by Watson-Crick base pairing and block mRNA translation through sequence-specific blockade. PMOs are resistant to nucleases and enzymes present in biologic fluids.

E. 5′(E)-Vinyl-Phosphonate (VP) Modification

5′-Vinyl-phosphonate modifications (metabolically stable phosphate mimics) have been reported to enhance the metabolic stability and the potency of oligonucleotides [31].

### II. COMPOSITIONS COMPRISING MIRNA MODULATORS

The compositions of the disclosure may comprise compounds of the disclosure combined with one or more carrier/targeting elements (e.g., any of the targeting agents described herein) to enhance specific cellular uptake, cellular distribution, and/or cellular activity of the compound. The compounds of the disclosure may directly or indirectly reprogram mesenchymal stem cells (ATMSCs) or white adipocytes (WAT) to become brown adipocytes (BAT) or beige adipocytes. The compounds can act on a target gene or an activator or repressor of a target gene, or on a target miRNA that directly or indirectly modulates the activity of a thermogenic regulator (e.g., a mitochondrial uncoupler or an activator or repressor thereof). As used herein, the term “mitochondrial uncoupler” refers to a protein (or the encoding nucleic acid) that can dissipate of the mitochondrial inner membrane proton gradient, thereby preventing the synthesis of ATP in the mitochondrion by oxidative phosphorylation. Exemplary mitochondrial uncouplers include UCP1, UCP2 and UCP3.

### III. METHODS OF TREATMENT

Methods of inhibiting miR-22 in a cell are disclosed herein. In some embodiments, a method of inhibiting miR-22 in a cell comprises administering to the cell a miR-22 antagonist, a molecule comprising an ONT agent alone or conjugated to a fatty acid or a short peptide. The cell may be an adipocyte, pre-adipocyte, fibroblast, hepatocyte or vascular endothelial cell. In some embodiments, the cell is an adipose tissue cell. In further embodiments, the adipose tissue cell is subcutaneous white adipose cell or brown adipose tissue cell.

The compositions disclosed herein may be used to positively affect a multitude of metabolic and thermogenesis processes. Some aspects of the present disclosure are directed towards a method for affecting body weight and fat mass reduction in a subject. Some aspects are directed towards increasing caloric expenditure in a subject. Some aspects are directed towards a method of decreasing total fat mass in a subject. Some aspects are directed towards a method of decreasing blood glucose levels in a subject. Some aspects are directed towards a method of decreasing blood insulin levels in a subject. Some aspects are directed towards a method of decreasing blood cholesterol levels in a subject. Some aspects are directed towards a method of decreasing blood leptin levels in a subject. Some embodiments are directed towards methods of maintaining insulin sensitivity in a subject. In some aspects, methods for converting white adipocytes to beige/brown adipocytes are disclosed. In some embodiments, methods for increasing lipolysis in a subject are disclosed. Some embodiments of the present disclosure are directed towards methods for increasing beta-oxidation of fatty acids in a subject. Some embodiments of the present disclosure are directed towards methods for reducing fatty infiltration of the liver. Some aspects of the present disclosure are directed towards methods for increasing thermogenesis in a subject. The methods comprise administering to a subject a composition comprising a miR-22-3p antagomir of the disclosure.

Such methods of treatment may be specifically tailored or modified, based on knowledge obtained from the field of pharmacogenomics. Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

ONTs can be tested in an appropriate animal model e.g., an obesity model including Diet-Induced Obesity (DIO) mice, ob/ob mice [32] and db/db mice [33]. For example, an ONT as described herein can be used in an animal model to determine the efficacy, toxicity, or side effects of treatment with said agent. Alternatively, a therapeutic agent can be used in an animal model to determine the mechanism of action of such an agent. For example, a compound of the disclosure can be used in an animal model to determine the efficacy, toxicity, or side effects of treatment with such an agent. Alternatively, a compound can be used in an animal model to determine the mechanism of action of such an agent.

The disclosure also provides a method for treating diabetes or pre-diabetes in a subject in need thereof comprising administering the subject one or more compounds disclosed herein. In some embodiments, the diabetes is type 2 diabetes.

The disclosure also provides a method for treating fatty infiltration of the liver in a subject in need thereof comprising administering the subject one or more compounds disclosed herein.

The disclosure also provides a method of causing fat loss in a subject in need thereof comprising administering the subject one or more compounds disclosed herein.

A compound of the disclosure modified for enhance uptake into cells (e.g., adipose cells) can be administered at a unit dose less than about 15 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of compound (e.g., about 4.4.times.1016 copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of compound per kg of bodyweight. The unit dose, for example, can be administered by injection (e.g., intravenous or intramuscular), an inhaled dose, or a topical application. Particularly preferred dosages are less than 2, 1, or 0.1 mg/kg of body weight.

Delivery of a compound of the disclosure directly to an organ or tissue (e.g., directly to adipose tissue) can be at a dosage on the order of about 0.00001 mg to about 3 mg per organ/tissue, or preferably about 0.0001-0.001 mg per organ/tissue, about 0.03-3.0 mg per organ/tissue, about 0.1-3.0 mg per organ/tissue or about 0.3-3.0 mg per organ/tissue. The dosage can be an amount effective to treat or prevent obesity. In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. In one embodiment, the effective dose is administered with other traditional therapeutic modalities.

In certain embodiment, a subject is administered an initial dose, and one or more maintenance doses of a composition. The maintenance dose or doses are generally lower than the initial dose, e.g., one-half less of the initial dose. A maintenance regimen can include treating the subject with a dose or doses ranging from 0.01 mg/kg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in conditions, e.g., changes in percentage of body fat. The dosage of the compound may either be increased in the event the patient does not respond significantly to current dosage levels, or the dose may be decreased if a decrease in body fat is observed, or if undesired side effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., sub-cutaneous, intravenous, intraperitoneal, intracisternal or intracapsular), or reservoir may be advisable. In one embodiment, a pharmaceutical composition includes a plurality of miRNA agent species. In another embodiment, the miRNA agent species has sequences that are non-overlapping and non-adjacent to another species with respect to a naturally occurring target sequence. In another embodiment, the plurality of miRNA agent species is specific for different naturally occurring target genes. In another embodiment, the miRNA agent is allele specific. In another embodiment, the plurality of miRNA agent species targets two or more SNP alleles (e.g., two, three, four, five, six, or more SNP alleles).

Following successful treatment, it may be desirable to have the patient undergo maintenance therapy to prevent the recurrence of the disease state, wherein the compound of the invention is administered in maintenance doses, ranging from 0.01 mg per kg to 100 mg per kg of body weight (see U.S. Pat. No. 6,107,094).

The “effective amount” of the compound is an amount sufficient to be effective in treating or preventing a disorder or to regulate a physiological condition in humans. The concentration or amount of miRNA agent administered will depend on the parameters determined for the agent and the method of administration, e.g., nasal, buccal, or pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a composition of the invention can include a single treatment or, preferably, can include a series of treatments. It will also be appreciated that the effective dosage of composition for treatment may increase or decrease over the course of a particular treatment. Changes in dosage may result and become apparent from the results of diagnostic assays as described herein. For example, the subject can be monitored after administering a compound of the disclosure. Based on information from the monitoring, an additional amount of the compound can be administered.

Dosing is dependent on severity and responsiveness of the disease condition to be treated, with the course of treatment lasting from several days to several months, or until a cure is obtained or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds and can generally be estimated based on EC50s found to be effective in in vitro and in vivo animal models. In some embodiments, the animal models include transgenic animals that express a human gene, e.g., a gene that produces a target mRNA (e.g., a thermogenic regulator). The transgenic animal can be deficient for the corresponding endogenous mRNA. In another embodiment, the composition for testing includes a miRNA analog that is complementary to a compound of the disclosure, at least in an internal region, to a sequence that is conserved between a nucleic acid sequence in the animal model and the target nucleic acid sequence in a human.

Several studies have reported successful mammalian dosing using miRNA agents. For example, Esau et al., 2006 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, nontoxic dose. Another study by Krutzfeldt et al., 2005, injected antagomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen et al. reported that efficient silencing of miR-122 was achieved in primates by three doses of 10 mg per kg LNA-antimiR, leading to a long-lasting and reversible decrease in total plasma cholesterol without any evidence for LNA-associated toxicities or histopathological changes in the study animals [34].

The compositions of the invention may be directly introduced into a cell (e.g., an adipocyte); or introduced extra-cellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

In certain embodiments, the methods described herein include co-administration of miRNA agents with other drugs or pharmaceuticals, e.g., compositions for modulating thermogenesis, compositions for treating diabetes, compositions for treating obesity. Compositions for modulating thermogenesis include beta-3 adrenergic receptor agonists, thyroid hormones, PPARG agonists, leptin, adiponectin, and orexin.

### IV. PHARMACEUTICAL PREPARATIONS

In one aspect, the methods disclosed herein can include the administration of pharmaceutical compositions and formulations comprising miRNA agents capable of modulating the activity of at least one thermogenic modulator.

In certain embodiments, the compositions are formulated with a pharmaceutically acceptable carrier. The pharmaceutical compositions and formulations can be administered parenterally, topically, by direct administration into the gastrointestinal tract (e.g., orally or rectally), or by local administration, such as by aerosol or trans-dermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The miRNA agents can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., oligonucleotides) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragées, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragée cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., oligonucleotides) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In certain embodiments, oil-based pharmaceuticals are used for administration of the miRNA agents. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

In certain embodiments, the pharmaceutical compositions and formulations are in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions comprise a paraffin oil, a sorbitan monooleate, an ethoxylated sorbitan monooleate and/or an ethoxylated sorbitan trioleate.

In certain embodiments, the pharmaceutical compositions and formulations are administered by in intranasal, intraocular and intravaginal routes including suppositories, insufflation, powders and aerosol formulations (for examples of steroid inhalants, see e.g., Rohatagi (1995) J. Clin. Pharmacol. 35: 1 187-1193; Tjwa (1995) Ann. Allergy Asthma Immunol. 75: 107-1 11). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In certain embodiments, the pharmaceutical compositions and formulations are delivered trans-dermally, by a topical route, formulated as applicator sticks, solutions, suspensions, emulsions, gels, creams, ointments, pastes, jellies, paints, powders, and aerosols.

In certain embodiments, the pharmaceutical compositions and formulations are delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In certain embodiments, the pharmaceutical compositions and formulations are parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In certain embodiments, the pharmaceutical compounds and formulations are lyophilized. Stable lyophilized formulations comprising an inhibitory nucleic acid can be made by lyophilizing a solution comprising a pharmaceutical and a bulking agent, e.g., mannitol, trehalose, raffinose, and sucrose or mixtures thereof. A process for preparing a stable lyophilized formulation can include lyophilizing a solution about 2.5 mg/mL nucleic acid, about 15 mg/mL sucrose, about 19 mg/mL NaCl, and a sodium citrate buffer having a pH greater than 5.5 but less than 6.5. See, e.g., U.S. 20040028670.

In certain embodiments, the pharmaceutical compositions and formulations are delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13: 293-306; Chonn (1995) Curr. Opin. Biotechnol. 6:698-708; Ostro (1989) Am. J. Hosp. Pharm. 46: 1576-1587.

The formulations can be administered for prophylactic and/or therapeutic treatments. In certain embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in certain embodiments, pharmaceutical compositions are administered in an amount sufficient to treat obesity in a subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51: 337-341; Fotherby (1996) Contraception 54:59-69; Johnson (1995) J. Pharm. Sci. 84: 1 144-1 146; Rohatagi (1995) Pharmazie 50:610-613; Brophy (1983) Eur. J. Clin. Pharmacol. 24: 103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods are correct and appropriate. Single or multiple administrations of formulations can be given depending on for example: the dosage and frequency as required and tolerated by the patient, the degree and amount of cholesterol homeostasis generated after each administration, and the like. The formulations should provide a sufficient quantity of active agent to effectively treat, prevent or ameliorate conditions, diseases or symptoms, e.g., treat obesity. In certain embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005. Examples.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—miR-22-3p Antagomirs

To test the efficacy of miR-22-3p antagomirs in the methods described herein, the nucleic acids and modified nucleic acids described in Tables 2-6.

The APT-200 family shown in Table 2 is made of 8 nucleotide-long miR-22-3p antagomirs with Lock Nucleic acid (LNA or 1n), DNA (d), Phosphorothioate (* or PS), and/or 5-Methylcytosine (5MeC) modifications.

TABLE 2
Locked Nucleic Acid (LNA) modified micro antagomiRs targeting the seed
region of human miR-22-3p

| Identifier | Length | Sequence | Comments |
|---|---|---|---|
| miR-22-3p | 22 | 5′-AAGCUGCCAGUUGAAGAACUGU-3′ | |
| | | (SEQ ID NO: 10) | |
| miR-22-3p | 22 | 5′-ACAGTTCTTCAACTGGCAGCTT-3′ | |
| AntagomiR | | (SEQ ID NO: 11) | |
| APT-110 | 18 | 5′-ln5MeC*-lnT*-dT*-mC*-dT*-dT*- | |
| | | ln5MeC*-dA*-dA*-mC*-dT*-lnG*-dG*- | |
| | | ln5Mec*-lnA*-dG*-ln5MeC*-lnT*-3′ | |
| | | (SEQ ID NO: 12) | |
| 2-9 8mer | 8 | 5′-TGGCAGCT-3′ (SEQ ID NO: 1) | |
| AntagomiR | | | |
| APT-200 | 8 | 5′-lnT-lnG-lnG-lnC-lnA-lnG-lnC-lnT-3′ | All Ln |
| APT-201 | 8 | 5′-lnT*-lnG*-lnG-lnC-lnA-lnG*-lnC*- | All Ln + 4* |
| | | lnT-3′ | |
| APT-202 | 8 | 5′-lnT*-lnG*-lnG*-lnC*-lnA*-lnG*- | All Ln + 7* |
| | | lnC*-lnT-3′ | |
| APT-203 | 8 | 5′-lnT*-lnG*-lnG-ln5MeC-lnA-lnG*- | All Ln + 4* + 2 5MeC |
| | | ln5MeC*-lnT-3′ | |
| APT-204 | 8 | 5′-lnT-lnG-dG-dC-dA-dG-lnC-lnT-3′ | 4 Ln + 4d |
| APT-205 | 8 | 5′-lnT*-lnG*-dG-dC-dA-dG*-lnC*-lnT-3′ | 4 Ln + 4d + 4* |
| APT-206 | 8 | 5′-lnT*-lnG*-dG*-dC*-dA*-dG*-lnC*- | 4 Ln + 4d + 7* |
| | | lnT-3′ | |
| APT-210 | 8 | 5′ dT*-lnG*-dG*-ln5MeC*-lnA*-dG*- | Short APT-110 + 7* |
| | | ln5MeC*-lnT-3′ | |
| APT-211 | 8 | 5′ dT*-lnG*-dG-ln5MeC-lnA-dG*- | Short APT-110 + 4* |
| | | ln5MeC*-lnT-3′ | |
| APT-212 | 8 | 5′-lnT*-lnG*-dG-ln5MeC-dA-dG*- | 3 ln + 3d + 2 |
| | | ln5MeC*-lnT-3 | ln5MeC + 4* |
| APT-213 | 8 | 5′-lnT*-lnG*-dG*-ln5MeC*-dA*-dG*- | 3 ln + 3d + 2 |
| | | ln5MeC*-lnT-3′ | ln5MeC + 7* |

ln = Locked Nucleic Acid; * = Phosphorothioate; 5MeC = 5-Methylcytosine, ln5MeC = Cytidine locked ribonucleotide with heterocyclic 5-methyl group; d = deoxy ribose, m-2′-O-Methylation of sugar

The APT-300 family shown in Table 3 is made of 8 to 12 nucleotide-long miR-22-3p antagomirs with Peptide Nucleic Acid (PNA) modifications and 5′ or 3′ end conjugation to a short peptide or Polyethylenimine.

TABLE 3
Peptide Nucleic Acid (PNA) modified micro antagomiRs targeting the seed
region of human miR-22-3p

| | | | SEQ ID | |
|---|---|---|---|---|
| Identifier | Length | Sequence | NO(S) | Comments |
| APT-300 | 8 | 5′-TGGCAGCT-3′ | 1 | All Pn 2-9 nt |
| APT-301 | 9 | 5′-CTGGCAGCT-3′ | 2 | All Pn 2-10 nt |
| APT-302 | 10 | 5′-ACTGGCAGCT-3′ | 3 | All Pn 2-11 nt |
| APT-303 | 11 | 5′-AACTGGCAGCT-3′ | 4 | All Pn 2-12 nt |
| APT-304 | 12 | 5′-CAACTGGCAGCT-3′ | 5 | All Pn 2-13 nt |
| APT-305 | 9 | 5′-CTGGCAGCT-3′-S-S-Hexarelin | 2 | All Pn + Hexarelin |
| APT-306 | 9 | 5′-CTGGCAGCT-3′-S-S-TSP-1 | 2 | All Pn + TSP-1 |
| APT-307 | 9 | 5′-CTGGCAGCT-3′-S-S-PHB | 2 | All Pn + PHB |
| APT-308 | 9 | 5′-CTGGCAGCT-3′-S-S-9R | 2 & 8 | All Pn + 9R |
| APT-309 | 9 | 5′-CTGGCAGCT-3′-S-S-4K | 2 & 9 | All Pn + 4K |
| APT-310 | 9 | 5′-CTGGCAGCT-3′-S-S-PEI | 2 | All Pn + PEI |
| APT-311 | 9 | 5′-Hexarelin-S-S-CTGGCAGCT-3′ | 2 | Hexarelin + All Pn |
| APT-312 | 9 | 5′-TSP-1-S-S-CTGGCAGCT-3′ | 2 | TSP-1 + All Pn |
| APT-313 | 9 | 5′-PHB-S-S-CTGGCAGCT-3′ | 2 | PHB + All Pn |
| APT-314 | 9 | 5′-9R-S-S-CTGGCAGCT-3′ | 8 & 2 | 9R + All Pn |
| APT-315 | 9 | 5′-4K-S-S-CTGGCAGCT-3′ | 9 & 2 | 4K + All Pn |
| APT-316 | 9 | 5′-PEI-S-S-CTGGCAGCT-3′ | 2 | PEI + All Pn |

Pn = Peptide Nucleic Acid; S-S = Disulfide bond (R-S-S-R); Hexarelin = His-D-2-Me-Trp-Ala-Trp-D-Phe-Lys-NH2; TSP-1 = GVITRIR (SEQ ID NO: 6); PHB = CKGGRAKDC (SEQ ID NO: 7); 9R = RRRRRRRRR (SEQ ID NO: 8); 4K = KKKK (SEQ ID NO: 9); PEI = Polyethylenimine

The APT-400 family shown in Table 4 is made of 8 nucleotide-long miR-22-3p antagomirs with Phosphorodiamidate Morpholino Oligonucleotide modifications, and 5′ or 3′ end conjugation to a short peptide or Polyethylenimine.

TABLE 4
Phosphorodiamidate Morpholino Oligonucleotide (PMO) modified micro
antagomiRs targeting the seed region of human miR-22-3p

| | | | SEQ ID | |
|---|---|---|---|---|
| Identifier | Length | Sequence | NO(S) | Comments |
| APT-400 | 8 | 5′-TGGCAGCT-3′ | 1 | All Po 2-9 nt |
| APT-401 | 9 | 5′-CTGGCAGCT-3′ | 2 | All Po 2-10 nt |
| APT-402 | 10 | 5′-ACTGGCAGCT-3′ | 3 | All Po 2-11 nt |
| APT-403 | 11 | 5′-AACTGGCAGCT-3′ | 4 | All Po 2-12 nt |
| APT-404 | 12 | 5′-CAACTGGCAGCT-3′ | 5 | All Po 2-13 nt |
| APT-405 | 9 | 5′-CTGGCAGCT-3′-S-S-Hexarelin | 2 | All Po + Hexarelin |
| APT-406 | 9 | 5′-CTGGCAGCT-3′-S-S-TSP-1 | 2 | All Po + TSP-1 |
| APT-407 | 9 | 5′-CTGGCAGCT-3′-S-S-PHB | 2 | All Po + PEIB |
| APT-408 | 9 | 5′-CTGGCAGCT-3′-S-S-9R | 2 & 8 | All Po + 9R |
| APT-409 | 9 | 5′-CTGGCAGCT-3′-S-S-4K | 2 & 9 | All Po + 4K |
| APT-410 | 9 | 5′-CTGGCAGCT-3′-S-S-PEI | 2 | All Po + PEI |
| APT-411 | 9 | 5′-Hexarelin-S-S-CTGGCAGCT-3′ | 2 | Hexarelin + All Po |
| APT-412 | 9 | 5′-TSP-1-S-S-CTGGCAGCT-3′ | 2 | TSP-1 + All Pn |
| APT-413 | 9 | 5′-PHB-S-S-CTGGCAGCT-3′ | 2 | PHB + All Pn |
| APT-414 | 9 | 5′-9R-S-S-CTGGCAGCT-3′ | 8 & 2 | 9R + All Pn |
| APT-415 | 9 | 5′-4K-S-S-CTGGCAGCT-3′ | 9 & 2 | 4K + All Pn |
| APT-416 | 9 | 5′-PEI-S-S-CTGGCAGCT-3′ | 2 | PEI + All Pn |

Po = Phosphorodiamidate Morpholino Oligonucleotide; S-S = Disulfide bond (R-S-S-R); Hexarelin = His-D-2-Me-Trp-Ala-Trp-D-Phe-Lys-NH2; TSP-1 = GVITRIR (SEQ ID NO: 6); PHB = CKGGRAKDC (SEQ ID NO: 7); 9R = RRRRRRRRR (SEQ ID NO: 8); 4K = KKKK (SEQ ID NO: 9); PEI = Polyethylenimine

The APT-500 family shown in Table 5 is made of 8 nucleotide-long miR-22-3p antagomirs with Ethylene-bridged Nucleic Acid (ENA), DNA (d), Phosphorothioate (*) and/or 5-Methylcytosine (5MeC), modifications.

TABLE 5
Ethylene-bridged Nucleic Acid (ENA) modified micro antagomiRs targeting the
seed region of human miR-22-3p

| Identifier | Length | Sequence | Comments | |
|---|---|---|---|---|
| APT-500 | 8 | 5′-enT-enG-enG-enC-enA-enG-enC-enT-3′ | All En | |
| APT-501 | 8 | 5′-enT*-enG*-enG-enC-enA-enG*-enC*-enT-3′ | All En + 4* | |
| APT-502 | 8 | 5′-enT*-enG*-enG*-enC*-enA*-enG*-enC*-enT-3′ | All En + 7* | |
| APT-503 | 8 | 5′-enT*-enG*-enG-en5MeC-enA-enG*-en5MeC*- | All En + 4* + 2 | |
| | | enT-3′ | 5MeC | |
| APT-504 | 8 | 5′-enT-enG-dG-dC-dA-dG-enC-enT-3′ | 4 En + 4d | |
| APT-505 | 8 | 5′-enT*-enG*-dG-dC-dA-dG*-enC*-enT-3′ | 4 | En + 4d + 4* |
| APT-506 | 8 | 5′-enT*-enG*-dG*-dC*-dA*-dG*-enC*-enT-3′ | 4 | En + 4d + 7* |

en = Ethylene-bridged Nucleic Acid; d = deoxy ribose; * = Phosphorothioate. 5MeC = 5-Methylcytosine

The APT-600 family shown in Table 6 is made of 8 nucleotide-long miR-22-3p antagomirs with 5′(E)-vinyl-phosphonate (VP), Lock Nucleic acid (LNA), Ethylene-bridged Nucleic Acid (ENA), 2′O-Methyl (2′-O-Me), 2′-Fluoro ((2′F) and/or 5-Methylcytosine (m5c) modifications.

TABLE 6
5′(E)-vinyl-phosphonate (VP) modified micro antagomiRs targeting the seed
region of human miR-22-3p

| Identifier | Length | Sequence | Comments |
|---|---|---|---|
| APT-601 | 8 | 5′-VP-lnT*-lnG*-dG-dC-dA-dG*-lnC*-lnT- | 5′VP + 4 Ln + 4d + 4* |
| | | 3′ | |
| APT-602 | 8 | 5′-VP-enT*-enG*-dG-dC-dA-dG*-enC*- | 5′VP + 4 En + 4d + 4* |
| | | enT-3′ | |
| APT-603 | 8 | 5′-VP-lnT*-lnG*-lnG-ln5MeC-lnA-lnG*- | 5′VP + All Ln + 4* + 2 |
| | | ln5MeC*-lnT-3′ | 5MeC |
| APT-604 | 8 | 5′-VP-enT*-enGenG-en5MeC-enA-enG*- | 5′VP + All En + 4* + 2 |
| | | en5MeC*-enT-3′ | 5MeC |
| APT-605 | 8 | 5′-VP- | 5′VP + 2′O-Me or 2′ |
| | | (mT)*(fG)*(mG)(fC)(mA)*(fG)*(mC)*(fT)- | F + 4* |
| | | 3′ | |
| APT-606 | 8 | 5′-VP- | 5′VP + 2′F or 2′ O- |
| | | (fT)*(mG)*(fG)(mC)(fA)(mG)*(fC)*(mT)- | m + 4* |
| | | 3′ | |

VP = 5′(E)-vinyl-phosphonate; ln = Locked Nucleic Acid; en = Ethylene-bridged Nucleic Acid; d = deoxy ribose; * = Phosphorothioate; 5MeC = 5-Methylcytosine; m = 2′ O-methyl modified ribose; f = 2′-Fluoride modified ribose.

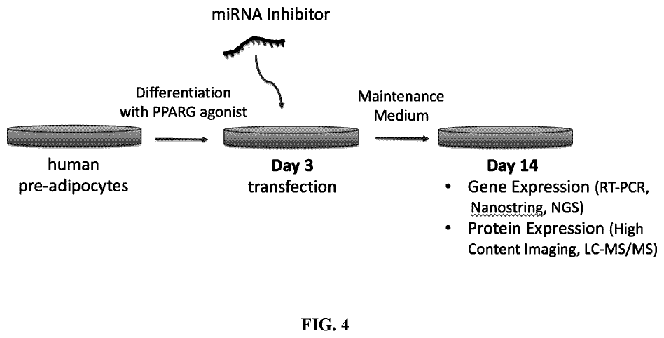

The inventor tested the ability of the specific miR-22-3p micro antagomirs to transform human fat storing white subcutaneous adipocytes into fat burning beige/brown adipocytes (browning effect). In vitro validation of these micro antagomir candidates was carried out in primary cultures of human subcutaneous adipocytes, the ultimate targets for an anti-obesity drug (clinical trial in a dish) following the experimental protocol shown in FIG. 4.

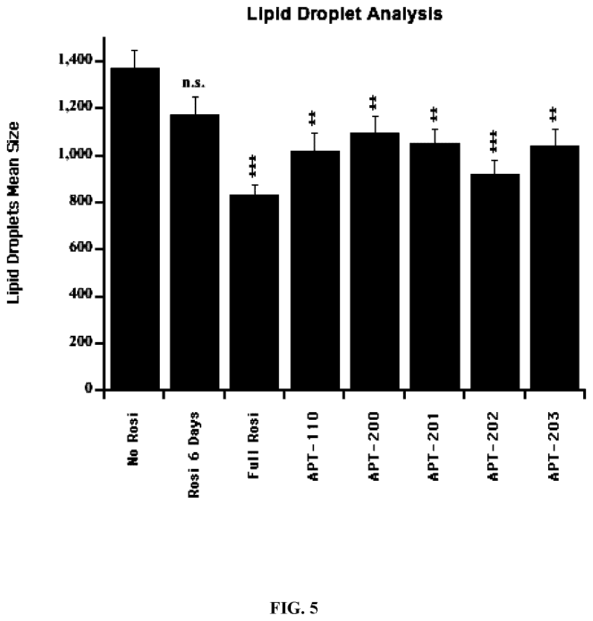

Adipocyte browning was assessed by measuring the size of lipid droplets present in the adipocytes in culture (confocal microscopy and ImageJ analysis). White adipocytes contain few large lipid droplets whereas beige/brown adipocytes contain a greater number of smaller lipid droplets. FIG. 5 is a representative example of this experiment. Members of the APT-200 series (8-mer LNA with PS and/or 2′ position or 5-Methylcytosine modifications) display a potent browning effect as illustrated by a significant size reduction of the lipid droplets after a single administration of the molecules at Day 3 of the experiment, especially APT-201 with only 4 PS residues and APT-202 with only 7 PS residues, as shown in FIG. 5

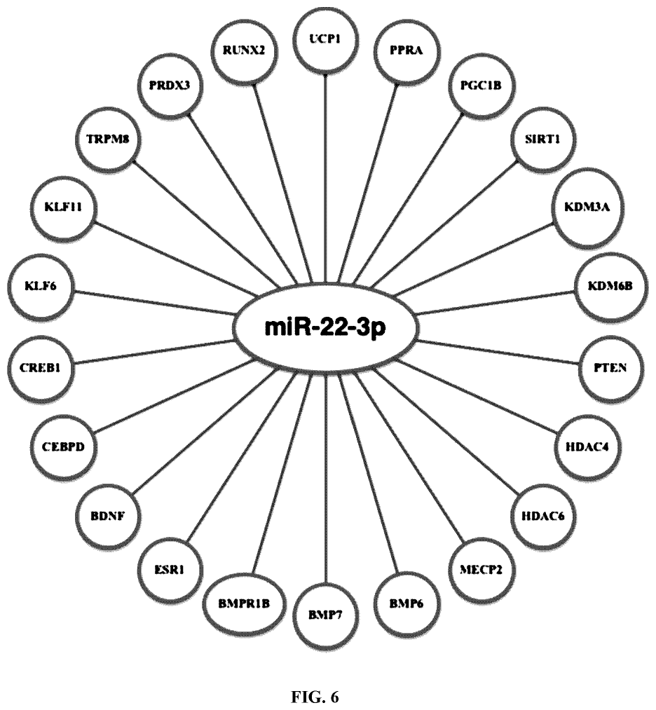

The inventor tested the ability of the specific miR-22-3p micro antagomirs to modify the expression of several target genes involved in lipid oxidation, mitochondrial functions, thermogenesis, glucose metabolism, adipocyte differentiation, inflammation and anti-oxidation modulated by miR-22-3p. FIG. 6 summarizes the target genes whose expression is modified by miR-22-3p.

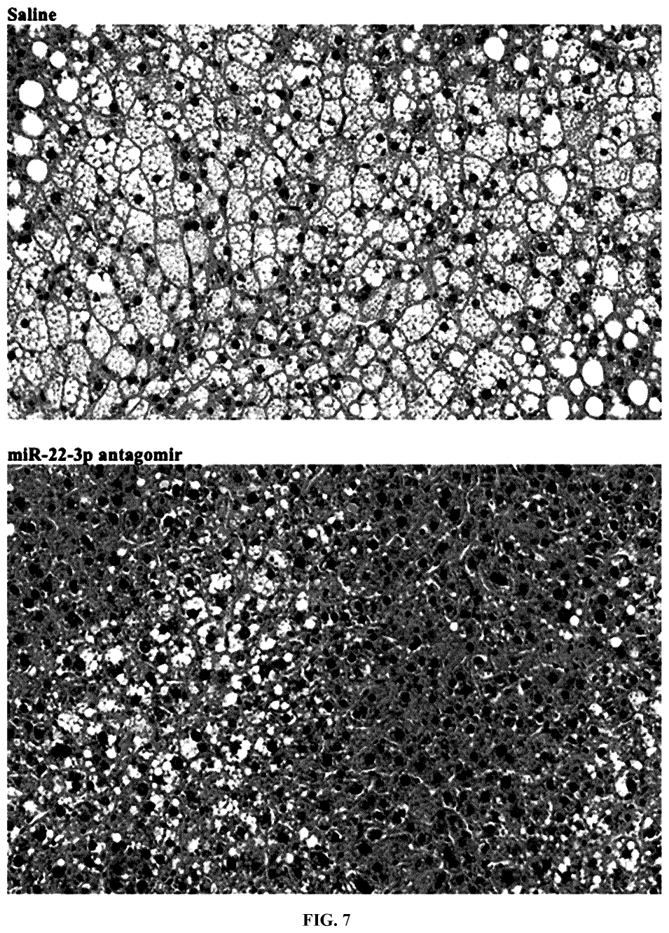

The inventor tested the ability of specific miR-22-3p micro antagomirs to reduce the accumulation of fat in the liver of DIO mice on a 60% high fat diet. FIG. 7 illustrates the dramatic reduction of fat accumulation in the livers of DIO mice on high fat diet treated with a miR-22-3p antagomir for 12 weeks.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Heymsfield, S. B. and T. A. Wadden, Mechanisms, Pathophysiology, and Management of Obesity. N Engl J Med, 2017. 376(3): p. 254-266.
2. WHO. Obesity and overweight. 2011; Available one the world wide web from: who.int/mediacentre/factsheets/fs311/en/index.html.
3. Ng, M., et al., Global, regional, and national prevalence of overweight and obesity in children and adults during 1980-2013: a systematic analysis for the Global Burden of Disease Study 2013. Lancet, 2014. 384(9945): p. 766-81.
4. Wilborn, C., et al., Obesity: prevalence, theories, medical consequences, management, and research directions. J Int Soc Sports Nutr, 2005. 2: p. 4-31.
5. Collaborators, G. B. D. O., et al., Health Effects of Overweight and Obesity in 195 Countries over 25 Years. N Engl J Med, 2017. 377(1): p. 13-27.
6. Wang, Y., et al., Will all Americans become overweight or obese? estimating the progression and cost of the US obesity epidemic. Obesity (Silver Spring), 2008. 16(10): p. 2323-30.
7. Grant, R. W. and V. D. Dixit, Adipose tissue as an immunological organ. Obesity (Silver Spring), 2015. 23(3): p. 512-8.
8. Gadde, K. M., et al., Obesity: Pathophysiology and Management. J Am Coll Cardiol, 2018. 71(1): p. 69-84.
9. Gadde, K. M., J. W. Apolzan, and H. R. Berthoud, Pharmacotherapy for Patients with Obesity. Clin Chem, 2018. 64(1): p. 118-129.
10. Younossi, Z., et al., Global burden of NAFLD and NASH: trends, predictions, risk factors and prevention. Nat Rev Gastroenterol Hepatol, 2018. 15(1): p. 11-20.
11. Hanson, A., D. Wilhelmsen, and J. K. DiStefano, The Role of Long Non-Coding RNAs (lncRNAs) in the Development and Progression of Fibrosis Associated with Nonalcoholic Fatty Liver Disease (NAFLD). Noncoding RNA, 2018. 4(3).
12. Sarwar, R., N. Pierce, and S. Koppe, Obesity and nonalcoholic fatty liver disease: current perspectives. Diabetes Metab Syndr Obes, 2018. 11: p. 533-542.
13. Buzzetti, E., M. Pinzani, and E. A. Tsochatzis, The multiple-hit pathogenesis of non-alcoholic fatty liver disease (NAFLD). Metabolism, 2016. 65(8): p. 1038-48.
14. Inui, M., G. Martello, and S. Piccolo, MicroRNA control of signal transduction. Nat Rev Mol Cell Biol, 2010. 11: p. 252-63.
15. Ha, M. and V. N. Kim, Regulation of microRNA biogenesis. Nat Rev Mol Cell Biol, 2014. 15(8): p. 509-24.
16. van Rooij, E. and S. Kauppinen, Development of microRNA therapeutics is coming of age. EMBO Mol Med, 2014. 6(7): p. 851-64.
17. Li, Z. and T. M. Rana, Therapeutic targeting of microRNAs: current status and future challenges. Nat Rev Drug Discov, 2014. 13(8): p. 622-38.
18. Rupaimoole, R. and F. J. Slack, MicroRNA therapeutics: towards a new era for the management of cancer and other diseases. Nat Rev Drug Discov, 2017. 16(3): p. 203-222.
19. Flierl, U., et al., Phosphorothioate backbone modifications of nucleotide-based drugs are potent platelet activators. J Exp Med, 2015. 212(2): p. 129-37.
20. Frazier, K. S., Antisense oligonucleotide therapies: the promise and the challenges from a toxicologic pathologist's perspective. Toxicol Pathol, 2015. 43(1): p. 78-89.
21. Yokoi, T. and M. Nakajima, microRNAs as mediators of drug toxicity. Annu Rev Pharmacol Toxicol, 2013. 53: p. 377-400.
22. Sewing, S., et al., Assessing single-stranded oligonucleotide drug-induced effects in vitro reveals key risk factors for thrombocytopenia. PLoS One, 2017. 12(11): p. e0187574.
23. Chi, X., P. Gatti, and T. Papoian, Safety of antisense oligonucleotide and siRNA-based therapeutics. Drug Discov Today, 2017. 22(5): p. 823-833.
24. Iannitti, T., J. C. Morales-Medina, and B. Palmieri, Phosphorothioate oligonucleotides: effectiveness and toxicity. Curr Drug Targets, 2014. 15(7): p. 663-73.
25. Hartmann, G., Nucleic Acid Immunity. Adv Immunol, 2017. 133: p. 121-169.
26. Obad, S., et al., Silencing of microRNA families by seed-targeting tiny LNAs. Nat Genet, 2011. 43: p. 371-8.
27. Rottiers, V., et al., Pharmacological inhibition of a microRNA family in nonhuman primates by a seed-targeting 8-mer antimiR. Sci Transl Med, 2013. 5(212): p. 212ra162.
28. Blechinger, J., et al., Short, terminally modified 2′-OMe RNAs as inhibitors of microRNA. Chem Commun (Camb), 2013. 49(67): p. 7397-9.
29. Khvorova, A. and J. K. Watts, The chemical evolution of oligonucleotide therapies of clinical utility. Nat Biotechnol, 2017. 35(3): p. 238-248.
30. Richard, D. and F. Picard, Brown fat biology and thermogenesis. Frontiers in bioscience: a journal and virtual library, 2011. 16: p. 1233-60.
31. Prakash, T. P., et al., Synergistic effect of phosphorothioate, 5′-vinylphosphonate and GalNAc modifications for enhancing activity of synthetic siRNA. Bioorg Med Chem Lett, 2016. 26(12): p. 2817-2820.
32. Lindstrom, P., The physiology of obese-hyperglycemic mice [ob/ob mice]. TheScientificWorldJournal, 2007. 7: p. 666-85.
33. Sharma, K., P. McCue, and S. R. Dunn, Diabetic kidney disease in the db/db mouse. American journal of physiology. Renal physiology, 2003. 284(6): p. F1138-44.
34. Elmen, J., et al., LNA-mediated microRNA silencing in non-human primates. Nature, 2008. 452: p. 896-9.

1-99. (canceled)

100. A modified nucleic acid comprising one of the following sequences targeting miR-22-3p:
(SEQ ID NO: 1)
TGGCAGCT,
(SEQ ID NO: 2)
CTGGCAGCT,
(SEQ ID NO: 3)
ACTGGCAGCT,
(SEQ ID NO: 4)
AACTGGCAGCT,
(SEQ ID NO: 5)
CAACTGGCAGCT,
(SEQ ID NO: 13)
TCAACTGGCAGCT,
(SEQ ID NO: 14)
TTCAACTGGCAGCT,
(SEQ ID NO: 15)
CTTCAACTGGCAGCT,
(SEQ ID NO: 16)
TCTTCAACTGGCAGCT,
(SEQ ID NO: 17)
TTCTTCAACTGGCAGCT,
or
(SEQ ID NO: 12)
CTTCTTCAACTGGCAGCT,
wherein at least one nucleotide is a locked nucleotide or an ethylene-bridged nucleotide.

101. The modified nucleic acid of claim 100, wherein the nucleic acid comprises one or more 2′-deoxy-ribose nucleotides.

102. The modified nucleic acid of claim 100, wherein the nucleic acid comprises one or more or more modified nucleobases.

103. The modified nucleic acid of claim 102 wherein the one or more modified nucleobases are one or more 5′-methylcytosines.

104. The nucleic acid of claim 100, wherein the modified nucleic acid comprises:
lnT-lnG-lnG-lnC-lnA-lnG-lnC-lnT;
lnT*-ln*G-lnG-lnC-lnA-lnG*-lnC*-lnT;
lnT*-lnG*-lnG*-lnC*-lnA*-lnG*-lnC*-lnT;
lnT*-lnG*-lnG-ln5MeC-lnA-lnG*-lnMeC*-lnT;
lnT-lnG-dG-dC-dA-dG-lnC-lnT;
lnT*-lnG*-dG-dC-dA-dG*-lnC*-lnT;
lnT*-lnG*-dG*-dC*-dA*-dG*-lnC*-lnT;
dT*-lnG*-dG*-ln5MeC*-lnA*-dG*-ln5MeC*-lnT;
dT*-lnG*-dG-ln5MeC-lnA-dG*-ln5MeC*-lnT;
lnT*-lnG*-dG-ln5MeC-dA-dG*-ln5MeC*-lnT;
or
lnT*-lnG*-dG*-ln5MeC*-dA*-dG*-ln5MeC*-lnT;
wherein lnX indicates that the nucleotide (X) is locked, 5MeC indicates a 5-methylcytosine nucleotide, ln5MeC indicates a Cytidine locked ribonucleotide with heterocyclic 5-methyl group; X* indicates a phosphorothioate modified nucleotide (X), and dX indicates a nucleotide (X) with a 2′-deoxy-ribose.

105. The nucleic acid of claim 100, wherein the modified nucleic acid comprises:
enT-enG-enG-enC-enA-enG-enC-enT;
enT*-enG*-enG-enC-enA-enG*-enC*-enT;
enT*-enG*-enG*-enC*-enA*-enG*-enC*-enT;
enT*-enG*-enG-en5MeC-enA-enG*-en5MeC*-enT;
enT-enG-dG-dC-dA-dG-enC-enT;
enT*-enG*-dG-dC-dA-dG*-enC*-enT;
or
enT*-enG*-dG*-dC*-dA*-dG*-enC*-enT;
wherein enX indicates that the nucleotide (X) is an ethylene-bridged nucleotide, X* indicates a phosphorothioate modified nucleotide (X), and 5MeC indicates a 5-methylcytosine nucleotide.

106. The nucleic acid of claim 100, wherein the modified nucleic acid comprises:
VP-lnT*-lnG*-dG-dC-dA-dG*-lnC*-lnT;
VP-enT*-enG*-dG-dC-dA-dG*-enC*-enT;
VP-lnT*-lnG*-lnG-ln5MeC-lnA-lnG*-ln5MeC*-lnT;
VP-enT*-enG*-enG-en5MeC-enA-enG*-en5MeC*-enT;
or
VP-(fT)*-(mG)*-(fG)-(mC)-(fA)*-(mG)*-(fC)*-(mT);
wherein VP indicates an 5′(E)-vinyl-phosphonate, mX indicates a nucleotide (X) with a 2′ O-methyl modified ribose, and fX indicates a nucleotide (X) with a 2′-fluoro modified ribose.

107. A method for antagonizing miR-22-3p in a cell or subject, the method comprising administering the modified nucleic acid of claim 100 to the cell or subject.

108. The method of claim 107, wherein the subject is overweight or obese or has a genetic or epigenetic predisposition to obesity.

109. The method of claim 107, wherein the modified nucleic acid is delivered trans-dermally to the subject.

110. A method of affecting weight loss in a subject comprising administering the modified nucleic acid of claim 100 to the subject.

111. A method of decreasing total fat mass in a subject comprising administering the modified nucleic acid of claim 100 to the subject.

112. A method for treating diabetes mellitus in a subject comprising administering the modified nucleic acid of claim 100 to the subject.

113. A method for treating non-alcoholic fatty liver disease (NAFLD) and/or metabolic disorders associated with NAFLD (Metabolic Dysfunction Associated Fatty Liver Disease, MAFLD) in a subject comprising administering the modified nucleic acid of claim 100 to the subject.

114. A method for treating one or more of liver steatosis, liver inflammation, liver fibrosis, hepatocellular mitochondrial dysfunction, and hepatocellular apoptosis in a subject, the method comprising administering the modified nucleic acid of claim 100 to the subject.

115. A peptide nucleic acid or phosphorodiamidate morpholino oligomer comprising one of the following sequences targeting miR-22-3p:
(SEQ ID NO: 1)
TGGCAGCT,
(SEQ ID NO: 2)
CTGGCAGCT,
(SEQ ID NO: 3)
ACTGGCAGCT,
(SEQ ID NO: 4)
AACTGGCAGCT,
(SEQ ID NO: 5)
CAACTGGCAGCT,
(SEQ ID NO: 13)
TCAACTGGCAGCT,
(SEQ ID NO: 14)
TTCAACTGGCAGCT,
(SEQ ID NO: 15)
CTTCAACTGGCAGCT,
(SEQ ID NO: 16)
TCTTCAACTGGCAGCT,
(SEQ ID NO: 17)
TTCTTCAACTGGCAGCT,
or
(SEQ ID NO: 12)
CTTCTTCAACTGGCAGCT.

116. The peptide nucleic acid or phosphorodiamidate morpholino oligomer of claim 115, wherein the peptide nucleic acid or phosphorodiamidate morpholino oligomer further comprises a modification at the carboxy or amino terminus of the peptide nucleic acid or at the 5′ end or the 3′ end of the phosphorodiamidate morpholino oligomer.

117. The peptide nucleic acid or phosphorodiamidate morpholino oligomer of claim 116, wherein the modification comprises one or more conjugated molecules.

118. The peptide nucleic acid or phosphorodiamidate morpholino oligomer of claim 117, wherein the one or more conjugated molecules comprise one or more of Hexarelin (L-Histidyl-2-methyl-D-tryptophyl-L-alanyl-L-tryptophyl-D-phenylalanyl-L-lysinamide), a Thrombospondin-1 (TSP-1) peptide comprising the amino acid sequence GVITRIR (SEQ ID NO:6), a prohibitin (PHB) peptide comprising the amino acid sequence CKGGRAKDC (SEQ ID NO:7), a nine Arginine (9R) peptide comprising the amino acid sequence RRRRRRRRR (SEQ ID NO:8), a four Lysine (4K) peptide comprising the amino acid sequence KKKK (SEQ ID NO:9), or polyethylenimine (C2H5N)n.

119. The peptide nucleic acid or phosphorodiamidate morpholino oligomer of claim 116 wherein the peptide nucleic acid or phosphorodiamidate morpholino oligomer is conjugated to a fatty acid.

120. The peptide nucleic acid or phosphorodiamidate morpholino oligomer of claim 119, wherein the fatty acid is a C10-35 chain fatty acid.

121. The peptide nucleic acid or phosphorodiamidate morpholino oligomer of claim 119, wherein the fatty acid comprises decanoic acid (C10), dodecanoic acid (C12), palmitic acid (C16), oleic acid (C16), stearic acid (C18), docosanoic acid (C22), docosahexanoic acid (C22), or dotriacontahexaenoic acid (C32).

122. The peptide nucleic acid or phosphorodiamidate morpholino oligomer of claim 115, wherein the peptide nucleic acid or phosphorodiamidate morpholino oligomer is a peptide nucleic acid selected from the group consisting of APT-300, APT-301, APT-302, APT-303, APT-304, APT-305, APT-306, APT-307, APT-308, APT-309, APT-310, APT-311, APT-312, APT-313, APT-314, APT-315, and APT-316.

123. The peptide nucleic acid or phosphorodiamidate morpholino oligomer of claim 115, wherein the peptide nucleic acid or phosphorodiamidate morpholino oligomer is a phosphorodiamidate morpholino oligomer selected from the group consisting of APT-400, APT-401, APT-402, APT-403, APT-404, APT-405, APT-406, APT-407, APT-408, APT-409, APT-410, APT-411, APT-412, APT-413, APT-414, APT-415, and APT-416.

124. A method for antagonizing miR-22-3p in a cell or subject, the method comprising administering the peptide nucleic acid or phosphorodiamidate morpholino oligomer of claim 118 to the cell or subject.

125. The method of claim 124, wherein the subject is overweight or obese or has a genetic or epigenetic predisposition to obesity.

126. The method of claim 124, wherein the modified nucleic acid is delivered trans-dermally to the subject.

127. A method for antagonizing miR-22-3p in a cell or subject, the method comprising administering the peptide nucleic acid or phosphorodiamidate morpholino oligomer of claim 119 to the cell or subject.

128. The method of claim 127, wherein the subject is overweight or obese or has a genetic or epigenetic predisposition to obesity.

129. The method of claim 127, wherein the modified nucleic acid is delivered trans-dermally to the subject.

130. A method of affecting weight loss in a subject comprising administering the peptide nucleic acid or phosphorodiamidate morpholino oligomer of claim 118 to the subject.

131. A method of affecting weight loss in a subject comprising administering the peptide nucleic acid or phosphorodiamidate morpholino oligomer of claim 119 to the subject.

132. A method of decreasing total fat mass in a subject comprising administering the peptide nucleic acid or phosphorodiamidate morpholino oligomer of claim 118 to the subject.

133. A method of decreasing total fat mass in a subject comprising administering the peptide nucleic acid or phosphorodiamidate morpholino oligomer of claim 119 to the subject.

134. A method for treating diabetes mellitus in a subject comprising administering the peptide nucleic acid or phosphorodiamidate morpholino oligomer of claim 118 to the subject.

135. A method for treating diabetes mellitus in a subject comprising administering the peptide nucleic acid or phosphorodiamidate morpholino oligomer of claim 119 to the subject.

136. A method for treating non-alcoholic fatty liver disease (NAFLD) and/or metabolic disorders associated with NAFLD (Metabolic Dysfunction Associated Fatty Liver Disease, MAFLD) in a subject comprising administering the peptide nucleic acid or phosphorodiamidate morpholino oligomer of claim 118 to the subject.

137. A method for treating non-alcoholic fatty liver disease (NAFLD) and/or metabolic disorders associated with NAFLD (Metabolic Dysfunction Associated Fatty Liver Disease, MAFLD) in a subject comprising administering the peptide nucleic acid or phosphorodiamidate morpholino oligomer of claim 119 to the subject.

138. A method for treating one or more of liver steatosis, liver inflammation, liver fibrosis, hepatocellular mitochondrial dysfunction, and hepatocellular apoptosis in a subject, the method comprising administering the peptide nucleic acid or phosphorodiamidate morpholino oligomer of any claim 118 to the subject.

139. A method for treating one or more of liver steatosis, liver inflammation, liver fibrosis, hepatocellular mitochondrial dysfunction, and hepatocellular apoptosis in a subject, the method comprising administering the peptide nucleic acid or phosphorodiamidate morpholino oligomer of claim 119 to the subject.
